SEQUENCE LISTING (35 sequences)

SEQ ID NO: 1 - protein, 4 aa, Artificial Sequence
  Description of Artificial Sequence Synthetic peptide
YADD

SEQ ID NO: 2 - RNA, 82 nt, Escherichia coli
ucauugagucuaaguuacguccuacggcuuuguuguaggucuccaacuggaauuacuauuuggagagcgauucccacgcgua

SEQ ID NO: 3 - DNA, 86 bp, Escherichia coli
gtcagaaaaaacgggtttcctggttggctcggagagcatcaggcgatgctctccgttccaacaaggaaaacagacagtaactcaga

SEQ ID NO: 4 - DNA, 56 bp, Artificial Sequence
  Description of Artificial Sequence Synthetic oligonucleotide
agatgcggtgttggtgtcgccagtctgactggcgacacaacagacagtaactcaga

SEQ ID NO: 5 - DNA, 33 bp, Artificial Sequence
  Description of Artificial Sequence Synthetic oligonucleotide
tgttgtgtcgccagtcagactggcgacacaaca

SEQ ID NO: 6 - DNA, 33 bp, Artificial Sequence
  Description of Artificial Sequence Synthetic oligonucleotide
tgttgtgtcgccagtctgactggcgacacaaca

SEQ ID NO: 7 - DNA, 57 bp, Artificial Sequence
  Description of Artificial Sequence Synthetic oligonucleotide
gtcagaaaaaacgggttgtcgccagtctgactggcgacaaacagacagtaactcaga

SEQ ID NO: 8 - DNA, 33 bp, Artificial Sequence
  Description of Artificial Sequence Synthetic oligonucleotide
ctgtttgtcgccagtcwgactggcgacaaacag

SEQ ID NO: 9 - DNA, 33 bp, Artificial Sequence
  Description of Artificial Sequence Synthetic oligonucleotide
tgtttgtcgccagtctgactggcgacaaacaga

SEQ ID NO: 10 - DNA, 33 bp, Artificial Sequence
  Description of Artificial Sequence Synthetic oligonucleotide
tgtttgtcgccagtcagactggcgacaaacaga

SEQ ID NO: 11 - DNA, 33 bp, Artificial Sequence
  Description of Artificial Sequence Synthetic oligonucleotide
tctgtttgtcgccagtctgactggcgacaaaca

SEQ ID NO: 12 - DNA, 33 bp, Artificial Sequence
  Description of Artificial Sequence Synthetic oligonucleotide
tctgtttgtcgccagtcagactggcgacaaaca

SEQ ID NO: 13 - DNA, 34 bp, Artificial Sequence
  Description of Artificial Sequence Synthetic oligonucleotide
gtctgtttgtcgccagtcagactggcgacaaaca

SEQ ID NO: 14 - DNA, 33 bp, Artificial Sequence
  Description of Artificial Sequence Synthetic oligonucleotide
ctgtttgtcgccagtctgactggcgacaaacag

SEQ ID NO: 15 - DNA, 33 bp, Artificial Sequence
  Description of Artificial Sequence Synthetic oligonucleotide
tgtctgtttgtcgccagtcagactggcgacaaa

SEQ ID NO: 16 - DNA, 34 bp, Artificial Sequence
  Description of Artificial Sequence Synthetic oligonucleotide
gtctgtttgtcgccagtctgactggcgacaaaca

SEQ ID NO: 17 - DNA, 33 bp, Artificial Sequence
  Description of Artificial Sequence Synthetic oligonucleotide
tgtcgccagtctgactggcgacaaacagacagt

SEQ ID NO: 18 - DNA, 33 bp, Artificial Sequence
  Description of Artificial Sequence Synthetic oligonucleotide
ctgtttgtcgccagtcagactggcgacaaacag

SEQ ID NO: 19 - DNA, 32 bp, Artificial Sequence
  Description of Artificial Sequence Synthetic oligonucleotide
gtttgtcgccagtctgactggcgacaaacaga

SEQ ID NO: 20 - DNA, 33 bp, Artificial Sequence
  Description of Artificial Sequence Synthetic oligonucleotide
tttgtcgccagtctgactggcgacaaacagaca

SEQ ID NO: 21 - DNA, 33 bp, Artificial Sequence
  Description of Artificial Sequence Synthetic oligonucleotide
tgtctgtttgtcgccagtctgactggcgacaaa

SEQ ID NO: 22 - DNA, 34 bp, Artificial Sequence
  Description of Artificial Sequence Synthetic oligonucleotide
tgtttgtcgccagtcagactggcgacaaacagac

SEQ ID NO: 23 - DNA, 57 bp, Artificial Sequence
  Description of Artificial Sequence Synthetic oligonucleotide
gtcagaaaaaacgggttgtcgccagtctgactggcgacaaacagcttgtaactcaga

SEQ ID NO: 24 - DNA, 33 bp, Artificial Sequence
  Description of Artificial Sequence Synthetic oligonucleotide
gctgtttgtcgccagtcwgactggcgacaaaca

SEQ ID NO: 25 - DNA, 33 bp, Artificial Sequence
  Description of Artificial Sequence Synthetic oligonucleotide
gctgtttgtcgccagtcagactggcgacaaaca

SEQ ID NO: 26 - DNA, 33 bp, Artificial Sequence
  Description of Artificial Sequence Synthetic oligonucleotide
gctgtttgtcgccagtctgactggcgacaaaca

SEQ ID NO: 27 - DNA, 32 bp, Artificial Sequence
  Description of Artificial Sequence Synthetic oligonucleotide
gctgtttgtcgccagtctgactggcgacaaac

SEQ ID NO: 28 - DNA, 34 bp, Artificial Sequence
  Description of Artificial Sequence Synthetic oligonucleotide
gctgtttgtcgccagtcagactggcgacaaacag

SEQ ID NO: 29 - DNA, 34 bp, Artificial Sequence
  Description of Artificial Sequence Synthetic oligonucleotide
gctgtttgtcgccagtctgactggcgacaaacag

SEQ ID NO: 30 - DNA, 33 bp, Artificial Sequence
  Description of Artificial Sequence Synthetic oligonucleotide
tgtttgtcgccagtctgactggcgacaaacagc

SEQ ID NO: 31 - DNA, 33 bp, Artificial Sequence
  Description of Artificial Sequence Synthetic oligonucleotide
tgtttgtcgccagtcagactggcgacaaacagc

SEQ ID NO: 32 - DNA, 32 bp, Artificial Sequence
  Description of Artificial Sequence Synthetic oligonucleotide
gctgtttgtcgccagtcagactggcgacaaac

SEQ ID NO: 33 - DNA, 33 bp, Artificial Sequence
  Description of Artificial Sequence Synthetic oligonucleotide
gctgtttgtcgccagtcagactggcgccaaaca

SEQ ID NO: 34 - DNA, 40 bp, Artificial Sequence
  Description of Artificial Sequence Synthetic oligonucleotide
aaacgggttgtcgccagtctgactggcgacaaacagcttg

SEQ ID NO: 35 - DNA, 57 bp, Artificial Sequence
  Description of Artificial Sequence Synthetic oligonucleotide
gtcagaaaaaacgggtggagaggttgctgcaacctctccattttcttgtaactcaga

(19) United States
(12) Patent Application Publication
(10) Pub. No.: US 2020/0115706 A1
(43) Pub. Date: Apr. 16, 2020

(54) Method of Recording Multiplexed Biological Information into a CRISPR Array Using a Retron

(71) Applicant: President and Fellows of Harvard College, Cambridge, MA (US)

(72) Inventors: Seth Lawler Shipman, Boston, MA (US); Jeffrey Matthew Nivala, Allston, MA (US); George M. Church, Brookline, MA (US); Max Schubert, Brookline, MA (US)

(21) Appl. No.: 16/499,889

(22) PCT Filed: Apr. 12, 2018
(86) PCT No.: PCT/US18/27344

§ 371 Date: Oct. 1, 2019

Related U.S. Application Data
(60) Provisional application No. 62/550,842, filed on Aug. 28, 2017.
(60) Provisional application No. 62/484,554, filed on Apr. 12, 2017.

(51) Int. Cl. C12N 15/10; C12N 9/22; C12N 15/11; C12N 15/70

(52) U.S. Cl. CPC C12N 15/1082; C12N 9/22; C12N 2800/80; C12N 15/70; C12N 2310/20; C12N 15/11

(57) ABSTRACT

This invention provides methods of altering a cell including providing the cell with a nucleic acid sequence encoding a Cas1 protein and/or a Cas2 protein of a CRISPR adaptation system, providing the cell with a CRISPR array nucleic acid sequence including a leader sequence and at least one repeat sequence, and providing the cell with one or more retron systems, wherein the cell expresses the Cas1 protein and/or the Cas2 protein.

### RELATED APPLICATION DATA

This application claims priority to U.S. Provisional Application No. 62/484,554 filed on Apr. 12, 2017 and U.S. Provisional Application No. 62/550,842 filed on Aug. 28, 2017, each of which is hereby incorporated herein by reference in its entirety for all purposes.

### STATEMENT OF GOVERNMENT INTERESTS

This invention was made with government support under Grant Nos. 4R01MH103910-04 and 5R01MH103910-04 awarded by National Institutes of Mental Health. The government has certain rights in the invention.

### SEQUENCE LISTING

The instant application contains a Sequence Listing which has been submitted electronically in ASCII format and is hereby incorporated by reference in its entirety. Said ASCII copy, created on Apr. 12, 2018, is named 010498_01085 WO_SL.txt and is 8,510 bytes in size.

### BACKGROUND

DNA is unmatched in its potential to encode, preserve, and propagate information (G. M. Church, Y. Gao, S. Kosuri, Next-generation digital information storage in DNA. Science 337, 1628 (2012); published online EpubSep 28 (10.1126/science.1226355)). The precipitous drop in DNA sequencing cost has now made it practical to read out this information at scale (J. Shendure, H. Ji, Next-generation DNA sequencing. Nat Biotechnol 26, 1135-1145 (2008); published online EpubOct (10.1038/nbt1486)). However, the ability to write arbitrary information into DNA, in particular within the genomes of living cells, has been restrained by a lack of biologically compatible recording systems that can exploit anything close to the full encoding capacity of nucleic acid space.

A number of approaches aimed at recording information within cells have been explored (D. R. Burrill, P. A. Silver, Making cellular memories. Cell 140, 13-18 (2010); published online EpubJan 8 (10.1016/j.cell.2009.12.034)). These systems can be broadly divided into those that encode events at the transcriptional level using feedback loops and toggles (N. T. Ingolia, A. W. Murray, Positive-feedback loops as a flexible biological module. Current biology: CB 17, 668-677 (2007); published online EpubApr 17 (10.1016/j.cub.2007.03.016), C. M. Ajo-Franklin, D. A. Drubin, J. A. Eskin, E. P. Gee, D. Landgraf, I. Phillips, P. A. Silver, Rational design of memory in eukaryotic cells. Genes & development 21, 2271-2276 (2007); published online EpubSep 15 (10.1101/gad.1586107), D. R. Burrill, M. C. Inniss, P. M. Boyle, P. A. Silver, Synthetic memory circuits for tracking human cell fate. Genes & development 26, 1486-1497 (2012); published online EpubJul 1 (10.1101/gad.189035.112), T. S. Gardner, C. R. Cantor, J. J. Collins, Construction of a genetic toggle switch in Escherichia coli. Nature 403, 339-342 (2000); published online EpubJan 20 (10.1038/35002131), D. Greber, M. D. El-Baba, M. Fussenegger, Intronically encoded siRNAs improve dynamic range of mammalian gene regulation systems and toggle switch. Nucleic acids research 36, e101 (2008); published online EpubSep (10.1093/nar/gkn443), M. R. Atkinson, M. A. Savageau, J. T. Myers, A. J. Ninfa, Development of genetic circuitry exhibiting toggle switch or oscillatory behavior in Escherichia coli. Cell 113, 597-607 (2003); published online EpubMay 30, H. Kobayashi, M. Kaern, M. Araki, K. Chung, T. S. Gardner, C. R. Cantor, J. J. Collins, Programmable cells: interfacing natural and engineered gene networks. Proc Natl Acad Sci USA 101, 8414-8419 (2004); published online EpubJun 1 (10.1073/pnas.0402940101), N. Vilaboa, M. Fenna, J. Munson, S. M. Roberts, R. Voellmy, Novel gene switches for targeted and timed expression of proteins of interest. Molecular therapy: the journal of the American Society of Gene Therapy 12, 290-298 (2005); published online EpubAug (10.1016/j.ymthe.2005.03.029), B. P. Kramer, M. Fussenegger, Hysteresis in a synthetic mammalian gene network. Proc Natl Acad Sci USA 102, 9517-9522 (2005); published online EpubJul 5 (10.1073/pnas.0500345102), D. R. Burrill, P. A. Silver, Synthetic circuit identifies subpopulations with sustained memory of DNA damage. Genes & development 25, 434-439 (2011); published online EpubMar 1 (10.1101/gad.1994911), M. Wu, R. Q. Su, X. Li, T. Ellis, Y. C. Lai, X. Wang, Engineering of regulated stochastic cell fate determination. Proc Natl Acad Sci USA 110, 10610-10615 (2013); published online EpubJun 25 (10.1073/pnas.1305423110)), versus those that encode information permanently into the genome, most often employing recombinases to store information via the orientation of DNA segments (T. S. Ham, S. K. Lee, J. D. Keasling, A. P. Arkin, Design and construction of a double inversion recombination switch for heritable sequential genetic memory. PLoS One 3, e2815 (2008)10.1371/journal.pone.0002815), T. S. Moon, E. J. Clarke, E. S. Groban, A. Tamsir, R. M. Clark, M. Eames, T. Kortemme, C. A. Voigt, Construction of a genetic multiplexer to toggle between chemosensory pathways in Escherichia coli. Journal of molecular biology 406, 215-227 (2011); published online EpubFeb 18 (10.1016/j.jmb.2010.12.019), J. Bonnet, P. Subsoontorn, D. Endy, Rewritable digital data storage in live cells via engineered control of recombination directionality. Proc Natl Acad Sci USA 109, 8884-8889 (2012); published online EpubJun 5 (10.1073/pnas.1202344109), L. Yang, A. A. Nielsen, J. Fernandez-Rodriguez, C. J. McClune, M. T. Laub, T. K. Lu, C. A. Voigt, Permanent genetic memory with >1-byte capacity. Nat Methods 11, 1261-1266 (2014); published online EpubDec (10.1038/nmeth.3147), P. Siuti, J. Yazbek, T. K. Lu, Synthetic circuits integrating logic and memory in living cells. Nat Biotechnol 31, 448-452 (2013); published online EpubMay (10.1038/nbt.2510)). While the majority of these systems are effectively binary, more recent efforts have also been made toward analogue recording systems (F. Farzadfard, T. K. Lu, Synthetic biology. Genomically encoded analog memory with precise in vivo DNA writing in living cell populations. Science 346, 1256272 (2014); published online EpubNov 14 (10.1126/science.1256272)) and digital counters (A. E. Friedland, T. K. Lu, X. Wang, D. Shi, G. Church, J. J. Collins, Synthetic gene networks that count. Science 324, 1199-1202 (2009); published online EpubMay 29 (10.1126/science.1172005)). Despite these efforts, the recording and genetic storage of little more than a single byte of information (L. Yang, A. A. Nielsen, J. Fernandez-Rodriguez, C. J. McClune, M. T. Laub, T. K. Lu, C. A. Voigt, Permanent genetic memory with >1-byte capacity. Nat Methods 11, 1261-1266 (2014); published online EpubDec (10.1038/nmeth.3147)) has remained out of reach.

Immunological memory is essential to an organism's adaptive immune response, and hence must be an efficient and robust form of recording molecular events into living cells. The CRISPR-Cas system is a recently understood form of adaptive immunity used by prokaryotes and archaea (R. Barrangou, C. Fremaux, H. Deveau, M. Richards, P. Boyaval, S. Moineau, D. A. Romero, P. Horvath, CRISPR provides acquired resistance against viruses in prokaryotes. Science 315, 1709-1712 (2007); published online EpubMar 23 (10.1126/science.1138140)). This system remembers past infections by storing short sequences of viral DNA within a genomic array. These acquired sequences are referred to as protospacers in their native viral context, and spacers once they are inserted into the CRISPR array. Importantly, new spacers are integrated into the CRISPR array ahead of older spacers (I. Yosef, M. G. Goren, U. Qimron, Proteins and DNA elements essential for the CRISPR adaptation process in Escherichia coli. Nucleic acids research 40, 5569-5576 (2012); published online EpubJul (10.1093/nar/gks216)). Over time, a long record of spacer sequences can be stored in the genomic array, arranged in the order in which they were acquired. Thus, the CRISPR array functions as a high capacity temporal memory bank of invading nucleic acids. However, there is a need for a CRISPR-Cas system that can direct recording of specific and arbitrary DNA sequences into the genome of prokaryotic and eukaryotic cells.

### SUMMARY

The present disclosure addresses this need and is based on the discovery that specific and arbitrary DNA sequences produced by one or more retron systems provided to and within a cell can be introduced and recorded into the genome of the cell. According to one aspect, a method of altering a cell is provided. The method includes providing the cell with one or more nucleic acid sequences encoding a Cas1 protein and/or a Cas2 protein of a CRISPR adaptation system, providing the cell with a CRISPR array nucleic acid sequence including a leader sequence and at least one repeat sequence, wherein the cell expresses the Cas1 protein and/or the Cas2 protein and wherein the CRISPR array nucleic acid sequence is within genomic DNA of the cell or on a plasmid. According to one aspect, the cell is also provided with a one or retron systems which are used to produce the DNA sequences referred to as protospacer sequences to be introduced into the CRISPR array. A retron system as described herein includes the components sufficient for one or more retrons to produce a double stranded oligonucleotide which is useful as a protospacer DNA sequence. More generally, the cell is provided with an exogenous DNA sequence which is transcribed into an RNA sequence. The RNA sequence is reverse transcribed in vivo into the protospacer DNA sequence and the protospacer DNA sequence is processed and inserted into the CRISPR array nucleic acid sequence using the Cas1 protein and/or the Cas2 protein to result in an inserted spacer sequence. According to one aspect, the method includes inserting two or more or a plurality of protospacer DNA sequences into a CRISPR array nucleic acid sequence such as by providing the cell with two or more or a plurality of exogenous DNA sequences which are correspondingly transcribed into two or more or plurality of RNA sequences, which are reverse transcribed in vivo into the two or more or plurality of protospacer DNA sequences, and two or more or a plurality of protospacer DNA sequences are inserted into the CRISPR array nucleic acid sequence using the Cas1 protein and/or the Cas2 protein to result in two or more or a plurality of inserted spacer sequences. According to one aspect, the step of reverse transcribing is accomplished using a retron system. According to one aspect, the step of reverse transcribing is accomplished using an exogenous retron system. According to one aspect, the step of reverse transcribing is accomplished using an exogenous retron system provided to a cell on a vector or where components of the retron system are provided to the cell on one or more vectors. The retron system produces single stranded DNA sequences which hybridize to produce a double stranded protospacer DNA sequence or the retron system produces a single stranded DNA which forms a hairpin to produce the double stranded protospacer DNA sequence.

According to one aspect, the protospacer sequence is a defined synthetic DNA. According to one aspect, the protospacer sequence includes a modified “AAG” protospacer adjacent motif (PAM). According to one aspect, the nucleic acid sequence encoding the Cas1 protein and/or a Cas2 protein is provided to the cell within a vector or within one or more vectors. According to one aspect, the retron system is provided to the cell within a vector or within one or more vectors. In certain embodiments, the cell is a prokaryotic or a eukaryotic cell. In one embodiment, the prokaryotic cell is E. coli. In another embodiment, the E. coli is BL21-AI. In one embodiment, the eukaryotic cell is a yeast cell, plant cell or a mammalian cell. In certain embodiments, the cell lacks endogenous Cas1 and Cas2 proteins. In certain embodiments, the cell lacks an endogenous retron system. In one embodiment, the nucleic acid sequence encoding the Cas1 protein and/or a Cas2 protein includes one or more inducible promoters for induction of expression of the Cas1 and/or Cas2 protein. In another embodiment, the nucleic acid sequence encoding the Cas1 protein and/or a Cas2 protein includes a first regulatory element operable in a eukaryotic cell. In one embodiment, the nucleic acid sequence encoding the Cas1 protein and/or a Cas2 protein is codon optimized for expression of Cas1 and/or Cas2 in a eukaryotic cell. According to one aspect, the protospacer is produced within the cell by the retron system within the cell and the cell is altered by inserting the protospacer sequence into the CRISPR array nucleic acid sequence to form an inserted spacer sequence.

According to another aspect, an engineered, non-naturally occurring cell is provided. In one embodiment, the cell includes one or more nucleic acid sequences encoding a Cast protein and/or a Cas2 protein of a CRISPR adaptation system wherein the cell expresses the Cas1 protein and/or the Cas 2 protein. In another embodiment, the cell includes a CRISPR array nucleic acid sequence including a leader sequence and at least one repeat sequence, wherein the CRISPR array nucleic acid sequence is inserted within genomic DNA of the cell or on a plasmid. According to one aspect, the cell further includes one or more retron systems which is used to produce the DNA sequences referred to as protospacer sequences to be introduced into the CRISPR array. In this manner, the cell produces the protospacer sequence and then the protospacer sequence is introduced into the CRISPR array to create an inserted spacer sequence.

According to one aspect, an engineered, non-naturally occurring cell is provided. In one embodiment, the cell includes one or more nucleic acid sequences encoding a Cas1 protein and/or a Cas2 protein of a CRISPR adaptation system, one or more retron systems, and a CRISPR array nucleic acid sequence including a leader sequence and at least one repeat sequence, wherein the cell expresses the Cas1 protein and/or the Cas 2 protein, and wherein the CRISPR array nucleic acid sequence is inserted within genomic DNA of the cell or on a plasmid.

According to another aspect, a method of inserting a target DNA sequence within genomic DNA of a cell is provided. In one embodiment, the method includes generating the target DNA sequence within a cell including one or more nucleic acid sequences encoding a Cas1 protein and/or a Cas2 protein of a CRISPR adaptation system and a CRISPR array nucleic acid sequence including a leader sequence and at least one repeat sequence, wherein the cell expresses the Cas1 protein and/or the Cas2 protein and wherein the CRISPR array nucleic acid sequence is within genomic DNA of the cell or on a plasmid, and wherein the target DNA sequence generated within the cell is under conditions within the cell wherein the Cas1 protein and/or the Cas2 protein processes the target DNA and the target DNA is inserted into the CRISPR array nucleic acid sequence adjacent a corresponding repeat sequence. In one embodiment, the target DNA sequence is a protospacer. In another embodiment, the target DNA protospacer is a defined synthetic DNA. In yet another embodiment, the target DNA sequence includes a modified “AAG” protospacer adjacent motif (PAM). In certain embodiments, the step of generating is repeated such that a plurality of target DNA sequences are inserted into the CRISPR array nucleic acid sequence at corresponding repeat sequences. According to one aspect, the step of generating one or more target DNA sequences is carried out by a retron system within the cell. In one embodiment, the one or more nucleic acid sequences encoding the Cas1 protein and/or a Cas2 protein is provided to the cell within a vector. In one embodiment, the one or more nucleic acid sequences encoding the retron system is provided to the cell within a vector.

According to one aspect, a nucleic acid storage system is provided. In one embodiment, the nucleic acid storage system includes an engineered, non-naturally occurring cell including one or more nucleic acid sequences encoding a Cas1 protein and/or a Cas2 protein of a CRISPR adaptation system, a CRISPR array nucleic acid sequence including a leader sequence and at least one repeat sequence, and one or more retron systems which is used to produce one or more protospacer DNA sequences to be introduced into the CRISPR array, wherein the cell expresses the Cas1 protein and/or the Cas2 protein and wherein the retron system produces the one or more protospacer DNA sequences, wherein the CRISPR array nucleic acid sequence is within genomic DNA of the cell or on a plasmid, wherein the one or more nucleic acid sequences encoding a Cas1 protein and/or a Cas2 protein is within genomic DNA of the cell or on one or more plasmids and/or wherein the one or more retron systems is on one or more plasmids. In one embodiment, at least one oligo nucleotide sequence comprises a protospacer inserted into the CRISPR array nucleic acid sequence.

According to another aspect, a method of recording molecular events into a cell is provided. In one embodiment, the method includes generating a DNA sequence or sequences containing information about the molecular events in the cell using a retron system within the cell wherein the cell includes one or more nucleic acid sequences encoding a Cas1 protein and/or a Cas2 protein of a CRISPR adaptation system and a CRISPR array nucleic acid sequence including a leader sequence and at least one repeat sequence, wherein the cell expresses the Cas1 protein and/or the Cas2 protein and wherein the CRISPR array nucleic acid sequence is within genomic DNA of the cell or on a plasmid, wherein the one or more nucleic acids encoding the Cas1 protein and/or the Cas2 protein is within genomic DNA of the cell or on a plasmid or wherein the one or more retron systems is within a plasmid, and wherein the DNA sequence is generated under conditions within the cell wherein the Cas1 protein and/or the Cas2 protein processes the DNA and the DNA is inserted into the CRISPR array nucleic acid sequence adjacent a corresponding repeat sequence. In certain embodiments, the step of generating is repeated such that a plurality of DNA sequences is inserted into the CRISPR array nucleic acid sequence at corresponding repeat sequences. In one embodiment, the DNA sequence includes a protospacer. In yet another embodiment, the protospacer is a defined synthetic DNA. In one embodiment, the DNA sequence includes a modified “AAG” protospacer adjacent motif (PAM). In certain embodiments, the molecular events comprise transcriptional dynamics, molecular interactions, signaling pathways, receptor modulation, calcium concentration, and electrical activity. In one embodiment, the recorded molecular events are decoded. In another embodiment, the decoding is by sequencing. In yet another embodiment, the decoding by sequencing comprises using the order information from pairs of acquired spacers in single cells to extrapolate and infer the order information of all recorded sequences within the entire population of cells. In one embodiment, the plurality of DNA sequences is recorded into a specific genomic locus of the cell in a temporal manner. In another embodiment, the DNA sequence is recorded into the genome of the cell in a sequence and/or orientation specific manner. In one embodiment, the DNA sequence includes a modified “AAG” protospacer adjacent motif (PAM). In another embodiment, the modified PAM is recognized by specific cas1 and/or cas2 mutants. In one embodiment, the protospacer is barcoded.

According to another aspect, a system for in vivo molecular recording is provided. In one embodiment, the system includes an engineered, non-naturally occurring cell including one or more nucleic acid sequences encoding a cas1 protein and/or a cas2 protein of a CRISPR adaptation system, one or more retron systems, and a CRISPR array nucleic acid sequence including a leader sequence and at least one repeat sequence, wherein the cell expresses the cast protein and/or the cas 2 protein and wherein the CRISPR array nucleic acid sequence is within genomic DNA of the cell or on a plasmid. In certain embodiments, the system records in single or multiple modalities. In one embodiment, the multiple modality recordation comprises altering Cas1 PAM recognition through directed evolution by specific cas1 or cas2 mutants.

According to one aspect, the disclosure provides a kit of directed recording of molecular events into a cell comprising an engineered, non-naturally occurring cell including a nucleic acid sequence encoding a cas 1 protein and/or a cas2 protein of a CRISPR adaptation system, one or more retron systems, and a CRISPR array nucleic acid sequence including a leader sequence and at least one repeat sequence, wherein the cell expresses the cas1 protein and/or the cas 2 protein and wherein the CRISPR array nucleic acid sequence is within genomic DNA of the cell or on a plasmid.

It is noted that in this disclosure and particularly in the claims and/or paragraphs, terms such as “comprises”, “comprised”, “comprising” and the like can have the meaning attributed to it in U.S. Patent law; e.g., they can mean “includes”, “included”, “including”, and the like; and that terms such as “consisting essentially of” and “consists essentially of” have the meaning ascribed to them in U.S. Patent law, e.g., they allow for elements not explicitly recited, but exclude elements that are found in the prior art or that affect a basic or novel characteristic of the invention.

Further features and advantages of certain embodiments of the present invention will become more fully apparent in the following description of embodiments and drawings thereof, and from the claims.

### BRIEF DESCRIPTION OF THE DRAWINGS

The patent or application file contains at least one drawing executed in color. Copies of this patent or patent application publication with color drawing(s) will be provided by the Office upon request and payment of the necessary fee. The foregoing and other features and advantages of the present embodiments will be more fully understood from the following detailed description of illustrative embodiments taken in conjunction with the accompanying drawings in which:

FIGS. 1A-1F depict the use of a retron system to generate protospacer DNA, which is acquired into the CRISPR array by Cas1 and Cas2 integrases. Modifications of an endogenous retron to create an msDNA compatible with the CRISPR acquisition system are shown. FIG. 1A depicts in schematic a retron plasmid including the msr, msd, and ret genes. FIG. 1B depicts an exemplary native retron known in the art as ec86. FIG. 1B discloses the RNA sequence as SEQ ID NO: 2 and the DNA sequence as SEQ ID NO: 3. FIG. 1C depicts the native ec86 structure redesigned to generate a DNA fragment compatible with CRISPR acquisition. FIG. 1C discloses SEQ ID NOS 4, 4 and 4-6, respectively, in order of appearance. FIG. 1D depicts data demonstrating that cells acquired the intended sequence into their CRISPR array. FIG. 1D discloses SEQ ID NOS 7, 8, 7, 7, 9-23, 24, 23, 23 and 25-33, respectively, in order of appearance. FIG. 1E discloses the RNA sequences as SEQ ID NOS 2 and 2 and the DNA sequences as SEQ ID NOS 3 and 23, respectively, in order of appearance.

FIG. 2A depicts an initial retron sequence (ec86 b3_v2) that was shown to be captured into a CRISPR array. FIG. 2A discloses the RNA sequence as SEQ ID NO: 2 and the DNA sequences as SEQ ID NOS 23, 34, 34 and 24, respectively, in order of appearance. FIG. 2B depicts a modified sequence (ec86 b3_v35) with nucleotides that differ from the sequence of FIG. 2A. FIG. 2B discloses the RNA sequence as SEQ ID NO: 2 and the DNA sequence as SEQ ID NO: 35. FIG. 2C depicts in schematic a first genetic element including inducible T7/lac promoters separately driving the msr- and msd encoding transcript and Cas1+2. A second genetic element is depicted with a separate and distinct (erythromycin-inducible) promoter on a different plasmid driving the ec86 reverse transcriptase. These elements are tested in BL21-Ai E. coli. FIG. 2D is a PAGE gel image showing both modified retron ssDNAs are produced by cells. FIG. 2E shows the timecourse of expression of the elements in FIG. 2C and sampling (16 hours of expression of the msr- and msd encoding transcript and Cas1+2, followed by 8 hours of expression of the reverse transcriptase, then 16 hours of growth, then samples are collected for sequencing). FIG. 2F is a graph showing that the two different msds are each detectable in the CRISPR array as new spacer sequences corresponding to the retron msd bases when separately induced. In the absence of the reverse transcriptase, no retron-derived spacer is acquired, indicating that neither the untranscribed plasmid element not the retron RNA are a significant source of spacer.

FIG. 3A depicts in schematic various genetic elements for producing a ssDNA from combined (cis) or separated (trans) modified forms of the retron. The bottom schematic indicates how the ssDNA can be expanded in length in the separated (trans) form by the addition of nucleotides toward the promoter from the msd. FIG. 3B is a gel image showing ssDNA produced from the various elements in FIG. 3A, including with insertions of various sizes. FIGS. 3C and D depict a construct where the position of the msr-encoding element or sequence and msd-encoding element or sequence are swapped compared to the wild-type positioning and each expanded to create a third protospacer using two separate retron-derived ssDNAs. FIG. 3E depicts data demonstrating spacer acquisition into the CRISPR array of all three retron-derived sequences, particularly showing the spacer created between the two sequences that creates an ‘AND’ logic gate.

### DETAILED DESCRIPTION

Embodiments of the present disclosure are directed to methods of altering a cell via CRISPR-Cas system. According to certain aspects, the Cas1-Cas2 complex integrates synthetic oligonucleotide spacers into genome of cells in vivo. The oligonucleotide spacers are produced within the cell as opposed to being exogenously supplied to the cell. According to one aspect, integration of synthetic oligo spacers via the Cas1-Cas2 complex can be harnessed as a multi-modal molecular recording system.

The ability to write a stable record of identified molecular events into a specific genomic locus would enable the examination of long cellular histories and have many applications, ranging from developmental biology to synthetic devices. According to one aspect, the disclosure provides that the type I-E CRISPR-Cas system of E. coli can acquire defined pieces of synthetic DNA that are generated within the cell, such as with a retron system. The retron system may be endogenous or exogenously provided. According to another aspect, the feature of CRISPR-Cas system of acquiring defined pieces of synthetic DNA produced within the cell is harnessed to generate records of specific DNA sequences with >100 bytes of information into a population of bacterial genomes. According to certain aspects, the disclosure provides applying directed evolution to alter PAM recognition of the Cas1-Cas2 complex. In certain embodiments, the disclosure provides expanded recordings into multiple modalities. In related embodiments, the disclosure provides using this system to reveal previously unknown aspects of spacer acquisition, which are fundamental to the CRISPR-Cas adaptation process. In certain other embodiments, the disclosure provides results that lay the foundations of a multimodal intracellular recording device with information capacity far exceeding any previously published synthetic biological memory system.

In one embodiment, the CRISPR-Cas system is harnessed to record specific and arbitrary DNA sequences into a bacterial genome wherein the DNA sequences are produced within the cell. According to one aspect, the cell is modified to include one or more retron systems. The retron system is used to produce the DNA sequences within the cell. In certain embodiments, a record of defined sequences, recorded over many days, and in multiple modalities can be generated. In certain other embodiments, this system is explored to elucidate fundamental aspects of native CRISPR-Cas spacer acquisition and leverage this knowledge to enhance the recording system.

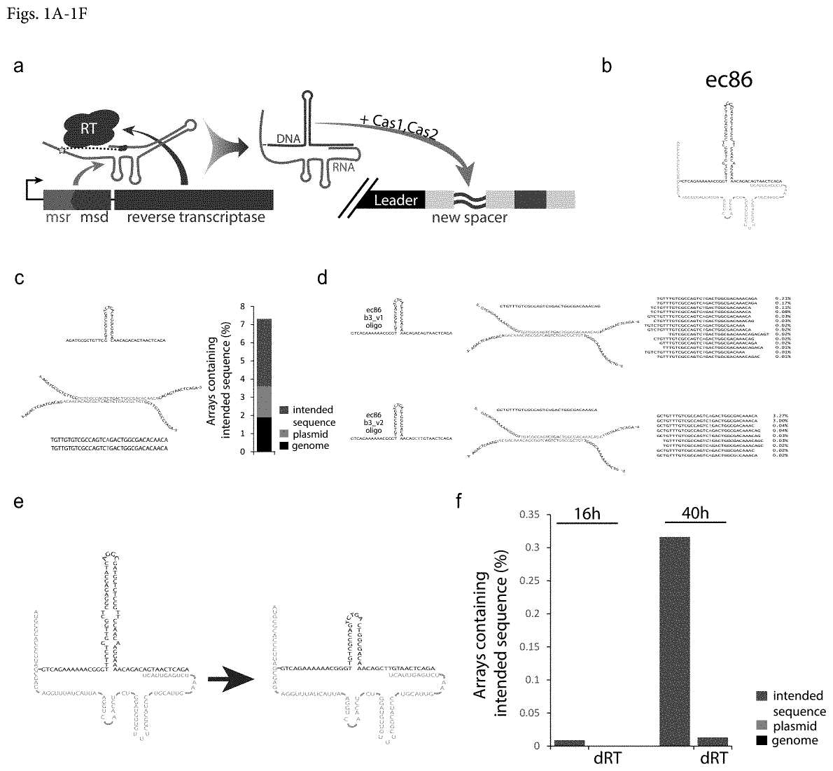

According to one aspect, the one or more oligonucleotide sequences to be inserted into the CRISPR array within a cell are produced in vivo by the cell. According to one aspect, a retron system is used to produce the one or more oligonucleotide sequences in vivo within a cell. According to one aspect, an exogenous dsDNA encoding the retron system is introduced into the cell. The retron system includes an msd/protospacer nucleic acid region and an msr nucleic acid region. The cell reverse transcribes the dsDNA into mRNA to produce an mRNA retron. The mRNA is reverse transcribed into msd DNA or protospacer DNA. According to one aspect, double stranded protospacer DNA is produced when two complementary msd sequences hybridize (two different msDNAs with complementary sequences, i.e. a Watson strand and a Crick strand, can hybridize to form the double stranded protospacer), or when an msd hybridizes with a second copy of the same msd (one msDNA can hybridize with another of the same sequence to form the double stranded protospacer (see FIG. 1C-1F), or when a double-stranded structure (such as a hairpin) is formed in a single msd (one msDNA can form an appropriate hairpin structure, providing the double stranded DNA).

Retrons are understood by those of skill in the art to be endogenous bacterial elements that generate ssDNA from a structured noncoding RNA transcript. See Lampson et al., Cytogenet Genome Res. 110 (104): 491-499 (2005) hereby incorporated by reference in its entirety. A retron is a distinct DNA sequence found in the genome of many bacteria species that codes for reverse transcriptase and a unique single-stranded DNA/RNA hybrid called multicopy single-stranded DNA (msDNA). Retron msr RNA is the non-coding RNA produced by retron elements and is the immediate precursor to the synthesis of msDNA. Internal base pairing creates various stem-loop/hairpin secondary structures in the msDNA. The retron msr RNA folds into a characteristic secondary structure that contains a conserved guanosine residue at the end of a stem loop. Synthesis of DNA by the retron-encoded reverse transcriptase (RT) results in the DNA/RNA chimera which is composed of small single-stranded DNA linked to small single-stranded RNA. The RNA strand is joined to the 5′ end of the DNA chain via a 2′-5′ phosphodiester linkage that occurs from the 2′ position of the conserved internal guanosine residue. The RT recognizes this secondary structure and uses a conserved guanosine residue in the msr as a priming site to reverse transcribe the msd sequence and produce a hybrid ssRNA-ssDNA molecule referred to as msDNA.

Retron elements may be about 2 kb long. They contain a single operon controlling the synthesis of an RNA transcript carrying three loci, msr, msd, and ret, that are involved in msDNA synthesis. The retron operon carries a promoter sequence P that controls the synthesis of an RNA transcript carrying the three loci, msr, msd, and ret. The ret gene product, a reverse transcriptase, processes the msd/msr portion of the RNA transcript into msDNA. Accordingly, the DNA portion of msDNA is encoded by the msd gene, the RNA portion is encoded by the msr gene, while the product of the ret gene is a reverse transcriptase similar to the RTs produced by retroviruses and other types of retroelements. Like other reverse transcriptases, the retron RT contains seven regions of conserved amino acids including a highly conserved tyr-ala-asp-asp (YADD) sequence (SEQ ID NO: 1) associated with the catalytic core. The ret gene product is responsible for processing the msd/msr portion of the RNA transcript into msDNA. According to the present disclosure, a single stranded DNA produced in vivo from a first retron may be hybridized with a complementary single stranded DNA produced in vivo from the same retron or a second retron or may form a hairpin structure and then is used as a protospacer sequence to be inserted into a CRISPR array as a spacer sequence. This aspect of the disclosure eliminates the introduction of an exogenous protospacer sequence using methods such as electroporation which can be disadvantageous in achieving sufficient levels of the protospacer sequence within a cell for introduction into a CRISPR array. The use of protospacers generated within the cell extends the in vivo molecular recording system from only capturing information known to a user, to capturing biological or environmental information that may be previously unknown to a user. For example, an msDNA protospacer sequence may be driven by a promoter that is downstream of a sensor pathway for a biological phenomenon or environmental toxin. The capture of that sequence records the event and stores it in the CRISPR array. If multiple msDNA protospacers are driven by different promoters, the activity of those promoters is recorded (along with anything that may be upstream of the promoters) as well as the relative order of promoter activity (based on the relative position of spacer sequences in the CRISPR array). At any point after the recording has taken place, one may sequence the array to determine whether a given biological or environmental event has taken place and the order of multiple events, given by the presence and relative position of msDNA-derived spacers in the CRISPR array.

The terms “polynucleotide”, “nucleotide”, “nucleotide sequence”, “nucleic acid” and “oligonucleotide” are used interchangeably. They refer to a polymeric form of nucleotides of any length, either deoxyribonucleotides or ribonucleotides, or analogs thereof. Polynucleotides may have any three dimensional structure, and may perform any function, known or unknown. The following are non limiting examples of polynucleotides: coding or non-coding regions of a gene or gene fragment, loci (locus) defined from linkage analysis, exons, introns, messenger RNA (mRNA), transfer RNA, ribosomal RNA, short interfering RNA (siRNA), short-hairpin RNA (shRNA), micro-RNA (miRNA), ribozymes, cDNA, recombinant polynucleotides, branched polynucleotides, plasmids, vectors, isolated DNA of any sequence, isolated RNA of any sequence, nucleic acid probes, and primers. A polynucleotide may comprise one or more modified nucleotides, such as methylated nucleotides and nucleotide analogs. If present, modifications to the nucleotide structure may be imparted before or after assembly of the polymer. The sequence of nucleotides may be interrupted by non nucleotide components. A polynucleotide may be further modified after polymerization, such as by conjugation with a labeling component.

The terms “non-naturally occurring” or “engineered” are used interchangeably and indicate the involvement of the hand of man. The terms, when referring to nucleic acid molecules or polypeptides mean that the nucleic acid molecule or the polypeptide is at least substantially free from at least one other component with which they are naturally associated in nature and as found in nature.

As used herein, “expression” refers to the process by which a polynucleotide is transcribed from a DNA template (such as into and mRNA or other RNA transcript) and/or the process by which a transcribed mRNA is subsequently translated into peptides, polypeptides, or proteins. Transcripts and encoded polypeptides may be collectively referred to as “gene product.” If the polynucleotide is derived from genomic DNA, expression may include splicing of the mRNA in a eukaryotic cell.

The terms “polypeptide”, “peptide” and “protein” are used interchangeably herein to refer to polymers of amino acids of any length. The polymer may be linear or branched, it may comprise modified amino acids, and it may be interrupted by non amino acids. The terms also encompass an amino acid polymer that has been modified; for example, disulfide bond formation, glycosylation, lipidation, acetylation, phosphorylation, or any other manipulation, such as conjugation with a labeling component. As used herein the term “amino acid” includes natural and/or unnatural or synthetic amino acids, including glycine and both the D or L optical isomers, and amino acid analogs and peptidomimetics.

In general, “a CRISPR adaptation system” refers collectively to transcripts and other elements involved in the expression of or directing the activity of CRISPR-associated (“Cas”) genes, including sequences encoding a Cas gene, and a CRISPR array nucleic acid sequence including a leader sequence and at least one repeat sequence. In some embodiments, one or more elements of a CRISPR adaption system is derived from a type I, type II, or type III CRISPR system. Cas1 and Cas2 are found in all three types of CRISPR-Cas systems, and they are involved in spacer acquisition. In the I-E system of E. coli, Cas1 and Cas2 form a complex where a Cas2 dimer bridges two Cas1 dimers. In this complex Cas2 performs a non-enzymatic scaffolding role, binding double-stranded fragments of invading DNA, while Cas1 binds the single-stranded flanks of the DNA and catalyzes their integration into CRISPR arrays.

In some embodiments, one or more elements of a CRISPR system is derived from a particular organism comprising an endogenous CRISPR system, such as Streptococcus pyogenes. In general, a CRISPR system is characterized by elements that promote the formation of a CRISPR complex at the site of a target sequence (also referred to as a protospacer in the context of an endogenous CRISPR system).

In some embodiments, a vector comprises a regulatory element operably linked to an enzyme-coding sequence encoding a CRISPR enzyme, such as a Cas protein. Non-limiting examples of Cas proteins include Cas1, Cas1B, Cas2, Cas3, Cas4, Cas5, Cas6, Cas7, Cas8, Cas9 (also known as Csn1 and Csx12), Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, homologs thereof, or modified versions thereof.

In certain embodiments, the disclosure provides protospacers that are adjacent to short (3-5 bp) DNA sequences termed protospacer adjacent motifs (PAM). The PAMs are important for type I and type II systems during acquisition. In type I and type II systems, protospacers are excised at positions adjacent to a PAM sequence, with the other end of the spacer is cut using a ruler mechanism, thus maintaining the regularity of the spacer size in the CRISPR array. The conservation of the PAM sequence differs between CRISPR-Cas systems and may be evolutionarily linked to Cas1 and the leader sequence.

In some embodiments, the disclosure provides for integration of defined synthetic DNA that is produced within a cell such as by using a retron system within the cell into a CRISPR array in a directional manner, occurring preferentially, but not exclusively, adjacent to the leader sequence. In the type I-E system from E. coli, it was demonstrated that the first direct repeat, adjacent to the leader sequence is copied, with the newly acquired spacer inserted between the first and second direct repeats.

In one embodiment, the protospacer is a defined synthetic DNA. In some embodiments, the defined synthetic DNA is at least 10, 20, 30, 40, or 50 nucleotides, or between 10-100, or between 20-90, or between 30-80, or between 40-70, or between 50-60, nucleotides in length.

In one embodiment, the oligo nucleotide sequence or the defined synthetic DNA includes a modified “AAG” protospacer adjacent motif (PAM).

In some embodiments, a regulatory element is operably linked to one or more elements of a CRISPR system so as to drive expression of the one or more elements of the CRISPR system. In general, CRISPRs (Clustered Regularly Interspaced Short Palindromic Repeats), also known as SPIDRs (SPacer Interspersed Direct Repeats), constitute a family of DNA loci that are usually specific to a particular bacterial species. The CRISPR locus comprises a distinct class of interspersed short sequence repeats (SSRs) that were recognized in E. coli (Ishino et al., J. Bacteriol., 169:5429-5433 [1987]; and Nakata et al., J. Bacteriol., 171:3553-3556 [1989]), and associated genes. Similar interspersed SSRs have been identified in Haloferax mediterranei, Streptococcus pyogenes, Anabaena, and Mycobacterium tuberculosis (See, Groenen et al., Mol. Microbiol., 10:1057-1065 [1993]; Hoe et al., Emerg. Infect. Dis., 5:254-263 [1999]; Masepohl et al., Biochim. Biophys. Acta 1307:26-30 [1996]; and Mojica et al., Mol. Microbiol., 17:85-93 [1995]). The CRISPR loci typically differ from other SSRs by the structure of the repeats, which have been termed short regularly spaced repeats (SRSRs) (Janssen et al., OMICS J. Integ. Biol., 6:23-33 [2002]; and Mojica et al., Mol. Microbiol., 36:244-246 [2000]). In general, the repeats are short elements that occur in clusters that are regularly spaced by unique intervening sequences with a substantially constant length (Mojica et al., [2000], supra). Although the repeat sequences are highly conserved between strains, the number of interspersed repeats and the sequences of the spacer regions typically differ from strain to strain (van Embden et al., J. Bacteriol., 182:2393-2401 [2000]). CRISPR loci have been identified in more than 40 prokaryotes (See e.g., Jansen et al., Mol. Microbiol., 43:1565-1575 [2002]; and Mojica et al., [2005]) including, but not limited to Aeropyrum, Pyrobaculum, Sulfolobus, Archaeoglobus, Halocarcula, Methanobacteriumn, Methanococcus, Methanosarcina, Methanopyrus, Pyrococcus, Picrophilus, Thernioplasnia, Corynebacterium, Mycobacterium, Streptomyces, Aquifrx, Porphvromonas, Chlorobium, Thermus, Bacillus, Listeria, Staphylococcus, Clostridium, Thermoanaerobacter, Mycoplasma, Fusobacterium, Azarcus, Chromobacterium, Neisseria, Nitrosomonas, Desulfovibrio, Geobacter, Myrococcus, Campylobacter, Wolinella, Acinetobacter, Erwinia, Escherichia, Legionella, Methylococcus, Pasteurella, Photobacterium, Salmonella, Xanthomonas, Yersinia, Treponema, and Thermotoga.

In some embodiments, an enzyme coding sequence encoding a CRISPR enzyme is codon optimized for expression in particular cells, such as eukaryotic cells. The eukaryotic cells may be those of or derived from a particular organism, such as a mammal, including but not limited to human, mouse, rat, rabbit, dog, or non-human primate. In general, codon optimization refers to a process of modifying a nucleic acid sequence for enhanced expression in the host cells of interest by replacing at least one codon (e.g. about or more than about 1, 2, 3, 4, 5, 10, 15, 20, 25, 50, or more codons) of the native sequence with codons that are more frequently or most frequently used in the genes of that host cell while maintaining the native amino acid sequence. Various species exhibit particular bias for certain codons of a particular amino acid. Codon bias (differences in codon usage between organisms) often correlates with the efficiency of translation of messenger RNA (mRNA), which is in turn believed to be dependent on, among other things, the properties of the codons being translated and the availability of particular transfer RNA (tRNA) molecules. The predominance of selected tRNAs in a cell is generally a reflection of the codons used most frequently in peptide synthesis. Accordingly, genes can be tailored for optimal gene expression in a given organism based on codon optimization. Codon usage tables are readily available, for example, at the “Codon Usage Database”, and these tables can be adapted in a number of ways. See Nakamura, Y., et al. “Codon usage tabulated from the international DNA sequence databases: status for the year 2000” Nucl. Acids Res. 28:292 (2000). Computer algorithms for codon optimizing a particular sequence for expression in a particular host cell are also available, such as Gene Forge (Aptagen; Jacobus, Pa.), are also available. In some embodiments, one or more codons (e.g. 1, 2, 3, 4, 5, 10, 15, 20, 25, 50, or more, or all codons) in a sequence encoding a CRISPR enzyme correspond to the most frequently used codon for a particular amino acid.

### Target DNA Sequence

The term “target DNA sequence” includes a nucleic acid sequence which is to be inserted into a CRISPR array nucleic acid sequence within the genomic DNA of the cell or on a plasmid according to methods described herein. The target DNA sequence may be expressed by the cell, for example, using a retron system within the cell as described herein. According to one aspect, the target DNA sequence is foreign to the cell, such that it is not a naturally occurring sequence produced by the cell other than the retron system. According to one aspect, the target DNA sequence is non-naturally occurring within the cell. According to another aspect, the target DNA sequence is synthetic. According to one aspect, the target DNA has a defined sequence.

### Foreign Nucleic Acids

Foreign nucleic acids (i.e. those which are not part of a cell's natural nucleic acid composition) may be introduced into a cell using any method known to those skilled in the art for such introduction. Such methods include transfection, transduction, viral transduction, microinjection, lipofection, nucleofection, nanoparticle bombardment, transformation, conjugation and the like. One of skill in the art will readily understand and adapt such methods using readily identifiable literature sources. According to one aspect, a foreign nucleic acid is exogenous to the cell. According to one aspect, a foreign nucleic acid is foreign, non-naturally occurring within the cell.

### Cells

Cells according to the present disclosure include any cell into which foreign nucleic acids can be introduced and expressed as described herein. It is to be understood that the basic concepts of the present disclosure described herein are not limited by cell type. Cells according to the present disclosure include eukaryotic cells, prokaryotic cells, animal cells, plant cells, fungal cells, archael cells, eubacterial cells and the like. Cells include eukaryotic cells such as yeast cells, plant cells, and animal cells. Particular cells include mammalian cells.

According to one aspect, the cell is a eukaryotic cell or a prokaryotic cell. According to one aspect, the cell is a yeast cell, bacterial cell, fungal cell, a plant cell or an animal cell. According to one aspect, the cell is a mammalian cell. According to one aspect, the cell is a human cell. According to one aspect, the cell is a stem cell whether adult or embryonic. According to one aspect, the cell is a pluripotent stem cell. According to one aspect, the cell is an induced pluripotent stem cell. According to one aspect, the cell is a human induced pluripotent stem cell. According to one aspect, the cell is in vitro, in vivo or ex vivo.

### Vectors

Vectors according to the present disclosure include those known in the art as being useful in delivering genetic material into a cell and would include regulators, promoters, nuclear localization signals (NLS), start codons, stop codons, a transgene etc., and any other genetic elements useful for integration and expression, as are known to those of skill in the art. The term “vector” includes a nucleic acid molecule capable of transporting another nucleic acid to which it has been linked. Vectors used to deliver the nucleic acids to cells as described herein include vectors known to those of skill in the art and used for such purposes. Certain exemplary vectors may be plasmids, lentiviruses or adeno-associated viruses known to those of skill in the art. Vectors include, but are not limited to, nucleic acid molecules that are single-stranded, double-stranded, or partially double-stranded; nucleic acid molecules that comprise one or more free ends, no free ends (e.g. circular); nucleic acid molecules that comprise DNA, RNA, or both; and other varieties of polynucleotides known in the art. One type of vector is a “plasmid,” which refers to a circular double stranded DNA loop into which additional DNA segments can be inserted, such as by standard molecular cloning techniques. Another type of vector is a viral vector, wherein virally-derived DNA or RNA sequences are present in the vector for packaging into a virus (e.g. retroviruses, lentiviruses, bacteriophages, herpes viruses, replication defective retroviruses, adenoviruses, replication defective adenoviruses, and adeno-associated viruses). Viral vectors also include polynucleotides carried by a virus for transfection into a host cell. Certain vectors are capable of autonomous replication in a host cell into which they are introduced (e.g. bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively linked. Such vectors are referred to herein as “expression vectors.” Common expression vectors of utility in recombinant DNA techniques are often in the form of plasmids. Recombinant expression vectors can comprise a nucleic acid of the invention in a form suitable for expression of the nucleic acid in a host cell, which means that the recombinant expression vectors include one or more regulatory elements, which may be selected on the basis of the host cells to be used for expression, that is operatively-linked to the nucleic acid sequence to be expressed. Within a recombinant expression vector, “operably linked” is intended to mean that the nucleotide sequence of interest is linked to the regulatory element(s) in a manner that allows for expression of the nucleotide sequence (e.g. in an in vitro transcription/translation system or in a host cell when the vector is introduced into the host cell).

Methods of non-viral delivery of nucleic acids or native DNA binding protein, native guide RNA or other native species include lipofection, microinjection, biolistics, virosomes, liposomes, immunoliposomes, polycation or lipid:nucleic acid conjugates, naked DNA, artificial virions, and agent-enhanced uptake of DNA. Lipofection is described in e.g., U.S. Pat. Nos. 5,049,386, 4,946,787; and 4,897,355) and lipofection reagents are sold commercially (e.g., Transfectam™ and Lipofectin™). Cationic and neutral lipids that are suitable for efficient receptor-recognition lipofection of polynucleotides include those of Felgner, WO 91/17424; WO 91/16024. Delivery can be to cells (e.g. in vitro or ex vivo administration) or target tissues (e.g. in vivo administration). The term native includes the protein, enzyme or guide RNA species itself and not the nucleic acid encoding the species.

### Regulatory Elements and Terminators and Tags

Regulatory elements are contemplated for use with the methods and constructs described herein. The term “regulatory element” is intended to include promoters, enhancers, internal ribosomal entry sites (IRES), and other expression control elements (e.g. transcription termination signals, such as polyadenylation signals and poly-U sequences). Such regulatory elements are described, for example, in Goeddel, GENE EXPRESSION TECHNOLOGY: METHODS IN ENZYMOLOGY 185, Academic Press, San Diego, Calif. (1990). Regulatory elements include those that direct constitutive expression of a nucleotide sequence in many types of host cell and those that direct expression of the nucleotide sequence only in certain host cells (e.g., tissue-specific regulatory sequences). A tissue-specific promoter may direct expression primarily in a desired tissue of interest, such as muscle, neuron, bone, skin, blood, specific organs (e.g. liver, pancreas), or particular cell types (e.g. lymphocytes). Regulatory elements may also direct expression in a temporal-dependent manner, such as in a cell-cycle dependent or developmental stage-dependent manner, which may or may not also be tissue or cell-type specific. In some embodiments, a vector may comprise one or more pol III promoter (e.g. 1, 2, 3, 4, 5, or more pol III promoters), one or more pol II promoters (e.g. 1, 2, 3, 4, 5, or more pol II promoters), one or more pol I promoters (e.g. 1, 2, 3, 4, 5, or more pol I promoters), or combinations thereof. Examples of pol III promoters include, but are not limited to, U6 and H1 promoters. Examples of pol II promoters include, but are not limited to, the retroviral Rous sarcoma virus (RSV) LTR promoter (optionally with the RSV enhancer), the cytomegalovirus (CMV) promoter (optionally with the CMV enhancer) [see, e.g., Boshart et al, Cell, 41:521-530 (1985)], the SV40 promoter, the dihydrofolate reductase promoter, the β-actin promoter, the phosphoglycerol kinase (PGK) promoter, and the EF1α promoter and Pol II promoters described herein. Also encompassed by the term “regulatory element” are enhancer elements, such as WPRE; CMV enhancers; the R-U5′ segment in LTR of HTLV-I (Mol. Cell. Biol., Vol. 8(1), p. 466-472, 1988); SV40 enhancer; and the intron sequence between exons 2 and 3 of rabbit β-globin (Proc. Natl. Acad. Sci. USA., Vol. 78(3), p. 1527-31, 1981). It will be appreciated by those skilled in the art that the design of the expression vector can depend on such factors as the choice of the host cell to be transformed, the level of expression desired, etc. A vector can be introduced into host cells to thereby produce transcripts, proteins, or peptides, including fusion proteins or peptides, encoded by nucleic acids as described herein (e.g., clustered regularly interspersed short palindromic repeats (CRISPR) transcripts, proteins, enzymes, mutant forms thereof, fusion proteins thereof, etc.).

Aspects of the methods described herein may make use of terminator sequences. A terminator sequence includes a section of nucleic acid sequence that marks the end of a gene or operon in genomic DNA during transcription. This sequence mediates transcriptional termination by providing signals in the newly synthesized mRNA that trigger processes which release the mRNA from the transcriptional complex. These processes include the direct interaction of the mRNA secondary structure with the complex and/or the indirect activities of recruited termination factors. Release of the transcriptional complex frees RNA polymerase and related transcriptional machinery to begin transcription of new mRNAs. Terminator sequences include those known in the art and identified and described herein.

Aspects of the methods described herein may make use of epitope tags and reporter gene sequences. Non-limiting examples of epitope tags include histidine (His) tags, V5 tags, FLAG tags, influenza hemagglutinin (HA) tags, Myc tags, VSV-G tags, and thioredoxin (Trx) tags. Examples of reporter genes include, but are not limited to, glutathione-S-transferase (GST), horseradish peroxidase (HRP), chloramphenicol acetyltransferase (CAT) beta-galactosidase, betaglucuronidase, luciferase, green fluorescent protein (GFP), HcRed, DsRed, cyan fluorescent protein (CFP), yellow fluorescent protein (YFP), and autofluorescent proteins including blue fluorescent protein (BFP).

The following examples are set forth as being representative of the present disclosure. These examples are not to be construed as limiting the scope of the present disclosure as these and other equivalent embodiments will be apparent in view of the present disclosure, figures and accompanying claims.

### Example I

### Materials and Methods

### Bacterial Strains, Plasmids, and Culturing Conditions

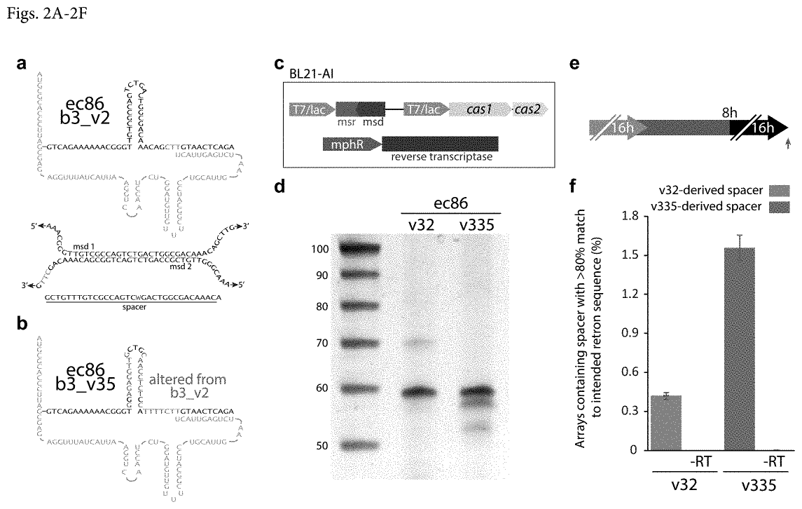

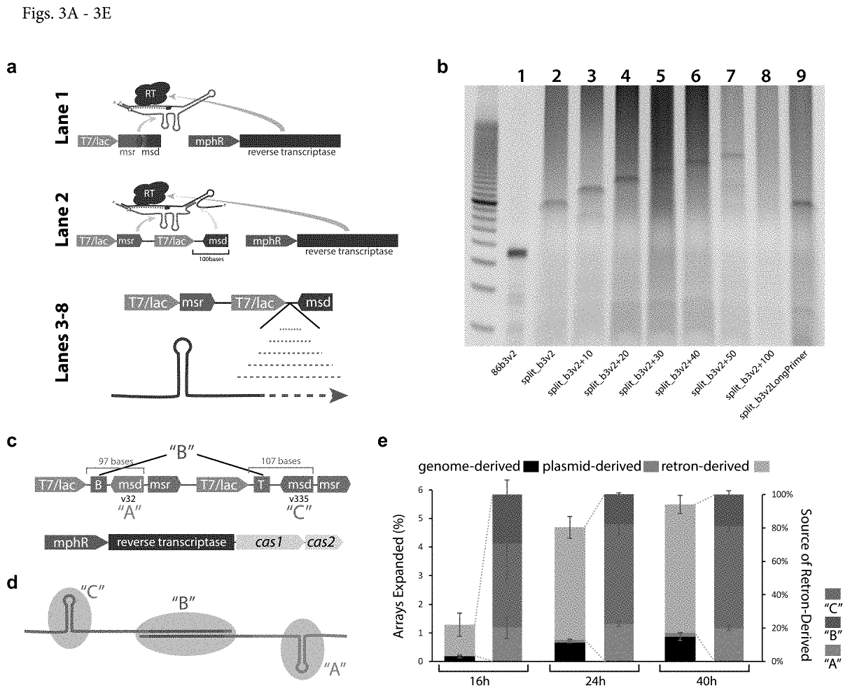

Experiments were carried out in BL21-AI E. coli (Thermo Fisher), containing an integrated, arabinose-inducible T7 polymerase, an endogenous CRISPR array, but no endogenous Cas1+2. For the electroporated protospacer experiments (see FIG. 1C and FIG. 1D), a plasmid encoding inducible (T7/lac) Cas1+2 (K-strain origin, pWUR1+2 a.k.a. pCas1+2) was transformed into cells prior to each experiment. Oligo protospacers were electroporated at 6.25 uM in water. For the retron-generated protospacer experiments (as described generally with respect to FIGS. 1A-1F and with respect to FIG. 1E and FIG. 1F in particular), a plasmid encoding Cas1+2 and a modified ec86 retron, both expressed by inducible (T7/lac) promoters (DUET-ec86(retron)-Cas1+2), was transformed into cells prior to each experiment. In the retron-based experiments depicted in FIGS. 2A, 2B, 2C, 2E and 2F and FIGS. 3A-3E, the reverse transcriptase was moved to a separate plasmid with an erythromycin-inducible promoter (mphR-ec86RT) (see Rogers et al., Nucleic Acids Res. 2015 Sep. 3; 43(15):7648-60. doi: 10.1093/nar/gkv616. Epub 2015 Jul. 7 hereby incorporated by reference in its entirety.) The msd and msr elements were expressed from an inducible T7 promoter, either together (DUET-T7-msr/msd-T7-Cas1+2) or separately (DUET-T7-msr-T7-msd). In the case of FIGS. 3C-3D, the endogenous arrangement of the msr and msd is swapped within a single transcript and the msd and msr are linked with a new four nucleotide loop. In this case, the reverse transcriptase, Cas1, and Cas2 are all expressed as a single operon from an erythromycin-inducible promoter on a separate plasmid. Cells containing plasmids were maintained in colonies on a plate at 4° C. for up to three weeks. Cells were grown in LB media at 34° C. and induced using IPTG, L-arabinose and/or erythromycin for the indicated durations.

### Electrophoretic Analysis of msd

To visualize the msd produced from modified retrons, bacteria were cultured for 16 hours in LB with all inducers necessary to express the msr-containing, msd-containing, and reverse-transcriptase-containing transcripts. A volume of 25 ml of culture was pelleted at 4° C., then prepared using a Plasmid Plus Midi Kit (Qiagen) without including RNase. The RNA was then digested using a combination of RNaseA and RNaseT1 and the resulting msd was purified using a ssDNA/RNA Clean & Concentrator kit (Zymo Research). The msd was visualized by running on a Novex TBE-Urea gel (Thermo Fisher) and post-staining with SYBR Gold (Thermo Fisher).

### Sequencing and Analysis

To analyze spacer acquisition, bacteria were lysed by heating to 95° C. for 5 minutes, then subjected to PCR of their genomic arrays using primers that flank the leader-repeat junction and additionally contain Illumina-compatible adapters. Spacer sequences were extracted bioinformatically based on the presence of flanking repeat sequences, and compared against pre-existing spacer sequences to determine the percentage of expanded arrays and the position and sequence of newly acquired spacers. New spacers were blasted (NCBI) against the genome and plasmid sequences and additionally compared against the intended protospacer sequence to determine the origin of the protospacer. This analysis was performed using custom written scripts in Python.

### Example II

### Results

FIG. 1A depicts in schematic a retron plasmid including the msr, msd, and ret genes. The msr and msd genes are transcribed into an msr/msd nocoding RNA transcript which is reverse transcribed into ssDNA to produce a protospacer sequence. The protospacer sequence is then used with Cas1 and/or Cas2 to insert a spacer sequence into the CRISPR array. According to one aspect, the protospacer sequence has a sequence and configuration which allows it to be processed for insertion into a CRISPR as is known in the art. As is known in the art, new acquisition of sequences into the CRISPR array requires the integrase complex of Cas1-Cas2 and double stranded DNA fragments to be acquired that include at least 23 complementary bases with at least 5 bases on the 3′ end of each strand that can be complementary or uncomplemented.

FIG. 1B depicts an exemplary native retron known in the art as ec86. See Lim et al., Cell 56, 891-904 (1989) hereby incorporated by reference in its entirety.

As shown in FIG. 1C, the native ec86 structure was redesigned to generate a DNA fragment compatible with CRISPR acquisition. In particular, the stem of the msDNA was shortened, non-complementary bases in the stem were removed, and the loop was modified so that two individual msDNAs with the same sequence could come together in the cell and form a complementary double-stranded fragment with a single mismatched base within a 22-24 base core duplexed region.

The oligonucleotides shown in FIG. 1C were electroporated into bacteria overexpressing Cas1-Cas2 and harboring a genomic CRISPR array. These cells acquired the intended sequence into their CRISPR array as indicated in FIG. 1C.

The oligonucleotides shown in FIG. 1C were then designed to be closer to the native ec86 in their flanking regions and were electroporated into bacteria overexpressing Cas1-Cas2 and harboring a genomic CRISPR array. The cells acquired the intended sequence into their CRISPR array, but that the addition of a protospacer adjacent motif (PAM, previously identified) increased the efficiency of acquisition as well as the reliability that the exact intended sequence would be acquired (rather than a sequence shifted by 1-6 bases). See FIG. 1D.

The modified msDNA structure shown in FIG. 1E was provided to the cell as an expressed retron. The retron and Cas1-Cas2 were overexpressed in bacteria harboring a genomic CRISPR array. The intended sequence was acquired into the genomic CRISPR array as shown in FIG. 1F. Notably, this was dependent on reverse transcription of the retron transcript, and thus generation of the msDNA in the cell. A mutant, inactive form of the reverse transciptase was tested resulting in loss of acquisition of the intended sequence as shown in FIG. 1F.

### Example III

### Multiplexing Multiple Protospacers

As described herein, aspects of the present disclosure are directed to inserting two or more or a plurality of protospacer DNA sequences into a CRISPR array nucleic acid sequence such as by providing the cell with two or more or a plurality of exogenous DNA sequences which are correspondingly transcribed into two or more or a plurality of RNA sequences, which are reverse transcribed in vivo into the two or more or plurality of protospacer DNA sequences, and two or more or a plurality of protospacer DNA sequences are inserted into the CRISPR array nucleic acid sequence using the Cas1 protein and/or the Cas2 protein to result in two or more or a plurality of inserted spacer sequences. According to one aspect, the step of reverse transcribing is accomplished using a retron system. According to one aspect, the cell is provided with a one or retron systems which are used to produce one or more protospacer DNA sequences to be introduced into the CRISPR array.

Multiple different retron sequences encoding multiple different ssDNA generating multiple different protospacer sequences are created. The creation of multiple different retron sequences encoding multiple different ssDNA generating multiple different protospacer sequences allows for the multiplexed introduction of multiple different protospacer sequences into a CRISPR array in a cell. The multiple different retron sequences include different msd sequences which produce different protospacer sequences. As described herein, different msd sequences may be driven by different promoter sequences, such as inducible promoters as described herein, to drive expression of the multiple and different msd. Multiple retron msd may be expressed at the same time or at different times to record individual and/or combinatorial activity of the promoters over time based on the spacer sequences that are captured into the CRISPR array. The different promoters may be downstream of sensors for biological activity or environmental conditions, such as a toxin.

According to one aspect, the msd sequence for a retron genetic element may be modified or designed or may differ between retron elements, i.e. a plurality of retron genetic elements, to provide a plurality of msd sequences for production of a plurality of different protospacer sequences. Transcription of the plurality of retron genetic elements having different msd sequences produced a plurality of different mRNA transcripts with each including a different msd transcript. The plurality of different mRNA transcripts are reverse transcribed by a reverse transcriptase to produce a plurality of different msDNA which then form a plurality of double stranded protospacer sequences for insertion into the CRISPR array by Cas1 and Cas2. As such, the disclosure contemplates insertion of multiple and different protospacer sequences into the CRISPR array in a multiplexed manner.

According to one aspect, methods and constructs as described above are provided for linking activation of a particular promoter to the insertion of a particular protospacer, insofar as cell may be provided with a plurality of different msd sequences, each with its own cognate promoter. The promoters may be induced or activated simultaneously or nonsimultaneously. Different promoters may be induced at different times resulting in the production of different protospacers over time. Analysis of the CRISPR array identifies whether a promoter has been activated insofar as a protospacer associated with the promoter has been inserted into the CRISPR array as a spacer sequence. A temporal analysis of which promoters are activated can be determined by analyzing the CRISPR array and determining the sequence of spacer sequences, which provides a timeline of msd activation to produce protospacers.

As described herein, one or more retron systems may be provided on one or more plasmids. According to certain aspects, the components of a retron system, i.e. msr, msd, and ret, can each have a separate and distinct promoter, i.e. a cognate promoter, such that each of msr, msd, and ret can be separately expressed. According to certain aspects, the components of a retron system, i.e. msr, msd, and ret, can each be provided on separate genetic elements having separate cognate promoter sequences such that each of msr, msd, and ret can be separately expressed. According to one aspect, the nucleic acid sequence encoding msr and msd can have a cognate promoter while the nucleic acid sequence encoding the ret gene can have a separate cognate promoter. According to one aspect, the msr and msd components of a retron system can be provided on a genetic element having a cognate promoter separate from a genetic element including the ret gene having a separate cognate promoter. According to this aspect, each of msr, msd, and ret can be transcribed into separate transcripts. The retron system may include a separate transcript for msr, a separate transcript for msd, and a separate transcript for ret. According to one aspect, the separate transcript for ret can be translated into a reverse transcriptase and the separate transcript for msr and the separate transcript for msd can combine to form a msr/msd transcript for reverse transcription by the reverse transcriptase. According to one aspect, the retron system may include a separate transcript including both msr and msd and a separate transcript including ret. According to one aspect, the retron system may include a separate transcript including both msr and msd and a separate transcript including ret where the msr and msd are arranged in the opposite order from the endogenous configuration and linked with a new four nucleotide loop. In this manner, the separate transcript for ret can be translated into a reverse transcriptase which will reverse transcribe the separate transcript including both msr and msd.

As shown in FIGS. 2A and 2B, two different exemplary internal DNA sequences can be used to generate different msds which form different protospacer sequences, each of which are capable of being processed by Cas1 and Cas2 and inserted into a CRISPR array. According to this aspect, two or more or a plurality of different exemplary internal DNA sequences can be designed and used with cognate promoters, such as inducible promters, to generate different msds which form different protospacer sequences, each of which are capable of being processed by Cas1 and Cas2 and inserted into a CRISPR array. FIG. 2A depicts an initial retron sequence (ec86 b3_v2) that was shown to be captured into a CRISPR array. FIG. 2B depicts a modified sequence (ec86 b3_v35) with nucleotides that differ from the initial sequence being shown in green. The bases encoding the PAM in each sequence (CTT in FIG. 2A and CTT in FIG. 2b) are shown in blue. Additional msd sequences can be designed.

FIGS. 2C and 2E depict in schematic a first genetic element BL21-Al including inducible T7/lac promoters separately driving the msr- and msd-encoding transcript and Cas1+2. A second genetic element is depicted with a separate and distinct (erythromycin-inducible) promoter on a different plasmid driving the ec86 reverse transcriptase. Cas1+2 and the msr/msd transcript are induced overnight, then the reverse transcriptase is induced for 8 hours. The cells are passaged and grown overnight without additional induction, then CRISPR arrays from the cells are sequenced. The two versions of the retron ssDNA were purified and the gel image of FIG. 2D demonstrates that both versions are able to be produced by the cell. FIG. 2F is a graph showing that the two different retron msds are each detectable in the CRISPR array as new spacer sequences corresponding to the retron msd bases when separately induced. Included in FIG. 2F is an RT control to demonstrate that the spacer sequence results from transcription and reverse-transcription of the target protospacer, and not from plasmid fragments.

A detailed experimental protocol is provided as follows. Cells containing the plasmids described were grown overnight in 3 ml of LB supplemented with L-arabinose (0.2% w/w/) and IPTG (1 mM) at 34° C. in a rotating drum. In the morning, cells were diluted (1:100) into fresh LB supplemented with erythromycin (450 μM) and grown for 8 hours at 34° C. in a rotating drum. Cells were then diluted again (1:100) into fresh LB and grown overnight in LB at 34° C. in a rotating drum. A sample of that culture was diluted 1:1 into water and prepared for sequencing as described in the materials and methods. New spacer origin was determined as described in the materials and methods.

As depicted in FIG. 3A, the retron can be arranged or designed to express the msr (RNA) and msd (DNA) transcripts separately using separate promoters. The msr and msd function in trans. The ret gene can also be expressed separately using a separate promoter. This separation eliminates the termination signal of the retron and allows for additional DNA bases to be added to the retron msd.

In FIG. 3A top, the arrangement or design is that from FIG. 2C, with one inducible promoter driving the overlapping msr and msd elements and a different inducible promoter driving the reverse transcriptase. The resulting purified msd is shown in Lane 1 of the PAGE gel in FIG. 3B. In FIG. 3A middle, the arrangement or design shows a version where the msr and msd are separated and expressed from two different inducible promoters. In this modified version, the msd does not terminate at the same location that it would in the endogenous arrangement. Rather, the msd continues back to the transcriptional start site. This extended msd is shown in Lane 2 of the PAGE gel in FIG. 3B. In FIG. 3A lower, additional stretches of DNA can be added between the promoter and msd-encoding bases on the plasmid which will elongate the reverse transcribed msd. Lanes 3-7 of the gel in FIG. 3B show insertions of increasing size, which yield msd sequences of increasing size. Lane 8 shows no band which may indicate a limit to which additional stretches of DNA can be added. Lane 9 shows a band for an extended msd using a long primer.

### Example IV

### msr/msd Inversion

Aspects of the present disclosure are directed to the rearrangement of wild-type ordering of retron elements. Wild-type retron elements include in series msr, msd and ret as depicted in FIG. 1A. Retrons can also be made by inverting the order of the msr and msd, which results in additional bases being reverse transcribed into DNA outside of the endogenous msd structure. The additional bases can be used to encode complementary sequences in two different retrons, i.e. two different msd sequences, that are co-expressed in order to form a double-stranded protospacer between the two different msd sequences. In this arrangement or design, three separate protospacer sequences are inserted into the CRISPR array, one from each individual retron msd and one that comes from the two retron msd sequences complementing each other. Accordingly, this design allows for the determination of whether both retron msd sequences are expressed insofar as expression of both leads to generation of a third protospacer sequence. This aspect forms the basis of using a retron to perform logic within a cell (e.g. an AND gate).

As depicted in FIG. 3C, the position of the msr-encoding element or sequence and msd-encoding element or sequence are swapped compared to the wild-type positioning insofar as the msd-encoding element is proximate to the promoter and precedes the msr-encoding element in the 5′ to 3′ direction. A nucleic acid loop sequence is inserted between the msd and msr and the effect is similar to Lanes 2-7 above, where the endogenous termination signal for the msd is removed, leading to an msd that is extended back to the transcriptional start site. In this case, two different msd sequences, with different internal sequences (the same as those shown in FIGS. 2A and 2B) were each driven by an inducible promoter on the same plasmid, and the extra bases outside of the endogenous msd structure were used to encode complementary bases between the two different retrons that would form a protospacer when duplexed. Thus, when both retrons are expressed, three separate sequence elements can form protospacers that can be captured into the CRISPR array: A first or “A” sequence generated within the stem of the second retron, a second or “B” sequence generated by the complemented regions of the two retrons, and a third or “C” sequence generated within the stem of the third sequence. The other elements of the system—the reverse transcriptase, Cas1, and Cas2—are expressed from a different inducible promoter on a different plasmid in a single designed operon, although each of the nucleic acids encoding the reverse transcriptase, the Cas1 and the Cas2 can be under the influence of a separate cognate promoter. As depicted by the data in FIG. 3E, when all elements of the system shown in FIGS. 3C and 3D are expressed, new spacer sequences are acquired into the CRISPR array, the majority of which are derived from the retron msd. Those new spacer sequences are drawn from each protospacer element, “A”, “B”, and “C” indicated in FIGS. 3C and 3D. FIG. 3E provides data for a number of replicates and also data for a 16 hour period, a 24 hour period and a 40 hour period.

According to one aspect, a DNA sequence is provided that includes in series a first msd and msr pair under influence of a first promoter, such as a T7/lac promoter, where the first msd region is proximal to the promoter and is followed by the msr when reading from a 5′ to 3′ direction. According to one aspect, the DNA sequence further includes in series a second msd and msr pair under influence of a second promoter, such as a T7/lac promoter. The first msd/msr pair is 5′ to the second msd/msr pair. The first msd encodes for a first complementary sequence. The second msd encodes for a second complementary sequence. When expressed, the first complementary sequence and the second complementary sequence hybridize to each other forming a protospacer sequence. The protospacer sequence is processed by Cas1 and Cas2 and is inserted into a CRISPR array as a spacer sequence.

A detailed experimental protocol is provided as follows. Cells containing the plasmids described were grown overnight in 3 ml of LB supplemented with L-arabinose (0.2% w/w/), IPTG (1 mM), and erythromycin (450 μM) at 34° C. in a rotating drum. A sample of that culture was diluted 1:1 into water and prepared for sequencing as described in the materials and methods. New spacer origin was determined as described in the materials and methods.

### Example V

### Embodiments

Aspects of the present disclosure are directed to a method of altering a cell including providing the cell with one or more nucleic acid sequences encoding a Cas1 protein and/or a Cas2 protein of a CRISPR adaptation system, providing the cell with a CRISPR array nucleic acid sequence including a leader sequence and at least one repeat sequence, wherein the CRISPR array nucleic acid sequence is within genomic DNA of the cell or on a plasmid, providing the cell with one or more retron systems which are used to produce protospacer DNA sequences to be introduced into the CRISPR array, wherein the cell expresses the Cast protein and/or the Cas2 protein, wherein the retron system produces the protospacer DNA sequence, and wherein the protospacer DNA sequence is processed and a spacer sequence is inserted into the CRISPR array nucleic acid sequence. According to one aspect, the protospacer is a defined synthetic DNA. According to one aspect, the protospacer sequence includes a modified “AAG” protospacer adjacent motif (PAM). According to one aspect, the nucleic acid sequence encoding the Cas1 protein and/or a Cas2 protein is provided to the cell within a vector. According to one aspect, the retron system is provided to the cell within a vector. According to one aspect, the cell is a prokaryotic or a eukaryotic cell. According to one aspect, the nucleic acid sequence encoding the Cas1 protein and/or a Cas2 protein comprises inducible promoters for induction of expression of the Cas1 and/or Cas2 protein.

According to one aspect, the cell is provided a plurality of retron systems which are used to produce different protospacer DNA sequences to be introduced into the CRISPR array, wherein the plurality of retron systems produce the different protospacer DNA sequences, and wherein the different protospacer DNA sequences are processed and spacer sequences are inserted into the CRISPR array nucleic acid sequence. According to one aspect, the retron system includes a first nucleic acid sequence comprising an msr sequence and an msd sequence under operation of a first cognate promoter and a second nucleic acid sequence comprising a ret sequence under operation of a second cognate promoter. According to one aspect, the retron system includes a first nucleic acid sequence comprising an msr sequence under operation of a first cognate promoter, a second nucleic acid sequence comprising an msd sequence under operation of a second cognate promoter and a third nucleic acid sequence comprising a ret sequence under operation of a third cognate promoter. According to one aspect, the retron system includes a first nucleic acid sequence comprising an msr sequence under operation of a first cognate promoter, a second nucleic acid sequence comprising an msd sequence under operation of a second cognate promoter and a third nucleic acid sequence comprising a ret sequence under operation of a third cognate promoter, wherein the second nucleic acid sequence includes an additional DNA sequence between the second cognate promoter and the msd sequence which is transcribed with the msd sequence. According to one aspect, methods further include providing the cell with a plurality of retron systems which are used to produce different protospacer DNA sequences to be introduced into the CRISPR array, wherein the plurality of retron systems produce the different protospacer DNA sequences, and wherein the different protospacer DNA sequences are processed and spacer sequences are inserted into the CRISPR array nucleic acid sequence, wherein each retron system of the plurality includes a first nucleic acid sequence comprising an msr sequence and an msd sequence under operation of a first cognate promoter and a second nucleic acid sequence comprising a ret sequence under operation of a second cognate promoter. According to one aspect, the first cognate promoter of each retron system is separately inducible. According to one aspect, the first cognate promoter of each retron system is separately inducible simultaneously or nonsimultaneously. According to one aspect, methods further include providing the cell with a plurality of retron systems which are used to produce different protospacer DNA sequences to be introduced into the CRISPR array, wherein the plurality of retron systems produce the different protospacer DNA sequences, and wherein the different protospacer DNA sequences are processed and spacer sequences are inserted into the CRISPR array nucleic acid sequence, wherein each retron system of the plurality includes a first nucleic acid sequence comprising an msr sequenced under operation of a first cognate promoter, a second nucleic acid sequence comprising an msd sequence under operation of a second cognate promoter and a third nucleic acid sequence comprising a ret sequence under operation of a third cognate promoter. According to one aspect, the second cognate promoter of each retron system is separately inducible. According to one aspect, the second cognate promoter of each retron system is separately inducible simultaneously or nonsimultaneously. According to one aspect, the second nucleic acid sequence includes an additional DNA sequence between the second cognate promoter and the msd sequence which is transcribed with the msd sequence.

Aspects of the present disclosure are directed to an engineered, non-naturally occurring cell including one or more nucleic acid sequences encoding a Cas1 protein and/or a Cas2 protein of a CRISPR adaptation system, a CRISPR array nucleic acid sequence including a leader sequence and at least one repeat sequence, and one or more retron systems which are used to produce protospacer DNA sequences to be introduced into the CRISPR array, wherein the CRISPR array nucleic acid sequence is within genomic DNA of the cell or on a plasmid, and wherein the cell expresses the Cas1 protein and/or the Cas 2 protein. According to one aspect, the cell includes at least one spacer sequence inserted into the CRISPR array nucleic acid sequence, which spacer sequence was derived from a corresponding protospacer sequence generated by the one or more retron systems. According to one aspect, the cell further includes a plurality of retron systems which are used to produce different protospacer DNA sequences to be introduced into the CRISPR array.

Aspects of the present disclosure are directed to method of inserting a target DNA sequence within genomic DNA of a cell including generating the target DNA sequence within the cell using one or more exogenous retron systems, wherein the cell includes a nucleic acid sequence encoding a Cas1 protein and/or a Cas2 protein of a CRISPR adaptation system and a CRISPR array nucleic acid sequence including a leader sequence and at least one repeat sequence, wherein the cell expresses the Cas1 protein and/or the Cas2 protein and wherein the CRISPR array nucleic acid sequence is within genomic DNA of the cell or on a plasmid, and wherein the target DNA sequence is generated under conditions within the cell wherein the Cas1 protein and/or the Cas2 protein processes the target DNA sequence and the target DNA sequence is inserted into the CRISPR array nucleic acid sequence adjacent a corresponding repeat sequence. According to one aspect, the target DNA sequence is a protospacer. According to one aspect, the target DNA sequence is a defined synthetic protospacer DNA sequence. According to one aspect, the target DNA sequence includes a modified “AAG” protospacer adjacent motif (PAM). According to one aspect, the step of generating is repeated such that a plurality of target DNA sequences are inserted into the CRISPR array nucleic acid sequence at corresponding repeat sequences. According to one aspect, the nucleic acid sequence encoding the Cas1 protein and/or a Cas2 protein is provided to the cell within a vector. According to one aspect, the cell is a prokaryotic or a eukaryotic cell. According to one aspect, methods further include inserting a plurality of different target DNA sequences within genomic DNA of a cell wherein the plurality of different target DNA sequences are generated within the cell using a plurality of exogenous retron systems, and wherein the Cas1 protein and/or the Cas2 protein processes the plurality of different target DNA sequences and the plurality of different target DNA sequences are inserted into the CRISPR array nucleic acid sequence adjacent a corresponding repeat sequence.

Aspects of the present disclosure are directed to a nucleic acid storage system including an engineered, non-naturally occurring cell including one or more nucleic acid sequences encoding a Cas1 protein and/or a Cas2 protein of a CRISPR adaptation system, a CRISPR array nucleic acid sequence including a leader sequence and at least one repeat sequence, and one or more retron systems which are used to produce protospacer DNA sequences to be processed and introduced into the CRISPR array, wherein the CRISPR array nucleic acid sequence is within genomic DNA of the cell or on a plasmid, and wherein the cell expresses the Cas1 protein and/or the Cas 2 protein. According to one aspect, at least one protospacer DNA sequence is generated by the one or more retron systems and is processed and a spacer sequence is inserted into the CRISPR array nucleic acid sequence. According to one aspect, the nucleic acid storage system further includes a plurality of retron systems which are used to produce different protospacer DNA sequences to be processed and introduced into the CRISPR array.

Aspects of the present disclosure are directed to a system for in vivo molecular recording including an engineered, non-naturally occurring cell including one or more nucleic acid sequences encoding a Cas1 protein and/or a Cas2 protein of a CRISPR adaptation system, a CRISPR array nucleic acid sequence including a leader sequence and at least one repeat sequence, and one or more retron systems which are used to produce protospacer DNA sequences to be processed and introduced into the CRISPR array, wherein the CRISPR array nucleic acid sequence is within genomic DNA of the cell or on a plasmid, and wherein the cell expresses the Cas1 protein and/or the Cas 2 protein. According to one aspect, the system further includes a plurality of retron systems which are used to produce different protospacer DNA sequences to be processed and introduced into the CRISPR array.

Aspects of the present disclosure are directed to a kit for in vivo molecular recording including in a first container, an engineered, non-naturally occurring cell including one or more nucleic acid sequences encoding a Cas1 protein and/or a Cas2 protein of a CRISPR adaptation system, a CRISPR array nucleic acid sequence including a leader sequence and at least one repeat sequence wherein the CRISPR array nucleic acid sequence is within genomic DNA of the cell or on a plasmid, in a second container, one or more retron systems to be supplied to the cell which are used to produce protospacer DNA sequences to be processed and introduced into the CRISPR array, and optional instructions for use. According to one aspect, the kit further includes in the second container, a plurality of retron systems to be supplied to the cell which are used to produce different protospacer DNA sequences to be processed and introduced into the CRISPR array.

Aspects of the present disclosure are directed to a method of altering a cell including providing the cell with one or more nucleic acid sequences encoding a Cas1 protein and/or a Cas2 protein of a CRISPR adaptation system, providing the cell with a CRISPR array nucleic acid sequence including a leader sequence and at least one repeat sequence, wherein the CRISPR array nucleic acid sequence is within genomic DNA of the cell or on a plasmid, providing the cell with a retron system which is used to produce different protospacer DNA sequences to be introduced into the CRISPR array, wherein the retron system includes (1) a first nucleic acid sequence comprising a first msd sequence 5′ to an msr sequence wherein the first msd sequence is proximal to and under operation of a first cognate promoter and further including a first complementary sequence between the first cognate promoter and the first msd sequence, (2) a second nucleic acid sequence comprising a second msd sequence 5′ to an msr sequence wherein the second msd sequence is proximal to and under operation of a second cognate promoter and further including a second complementary sequence between the second cognate promoter and the second msd sequence, wherein the first msd sequence is different from the second msd sequence and wherein the first complementary sequence and the second complementary sequence are complementary to each other, and (3) a third nucleic acid comprising a ret sequence under operation of a third cognate promoter, wherein the cell expresses the Cas1 protein and/or the Cas2 protein, wherein the retron system produces a first protospacer DNA sequence corresponding to the first msd sequence, a second protospacer DNA sequence corresponding to the second msd sequence, and a third protospacer sequence corresponding to the first complementary sequence and the second complementary sequence hybridized to each other, wherein the first, second and third protospacer DNA sequences are processed and spacer sequences are inserted into the CRISPR array nucleic acid sequence. According to one aspect, the first cognate promoter and the second cognate promoter of the retron system are separately inducible. According to one aspect, the first cognate promoter and the second cognate promoter of the retron system are separately inducible simultaneously or nonsimultaneously. According to one aspect, the first, second and third protospacer DNA sequences are defined synthetic DNA. According to one aspect, the first, second and third protospacer DNA sequences include a modified “AAG” protospacer adjacent motif (PAM). According to one aspect, the one or more nucleic acid sequences encoding the Cas1 protein and/or a Cas2 protein is provided to the cell within a vector. According to one aspect, the retron system is provided to the cell within a vector. According to one aspect, the cell is a prokaryotic or a eukaryotic cell.

Aspects of the present disclosure are directed to an engineered, non-naturally occurring cell including one or more nucleic acid sequences encoding a Cas1 protein and/or a Cas2 protein of a CRISPR adaptation system, a CRISPR array nucleic acid sequence including a leader sequence and at least one repeat sequence, wherein the CRISPR array nucleic acid sequence is within genomic DNA of the cell or on a plasmid, a retron system which is used to produce different protospacer DNA sequences to be introduced into the CRISPR array, wherein the retron system includes (1) a first nucleic acid sequence comprising a first msd sequence 5′ to an msr sequence wherein the first msd sequence is proximal to and under operation of a first cognate promoter and further including a first complementary sequence between the first cognate promoter and the first msd sequence, (2) a second nucleic acid sequence comprising a second msd sequence 5′ to an msr sequence wherein the second msd sequence is proximal to and under operation of a second cognate promoter and further including a second complementary sequence between the second cognate promoter and the second msd sequence, wherein the first msd sequence is different from the second msd sequence and wherein the first complementary sequence and the second complementary sequence are complementary to each other, and (3) a third nucleic acid comprising a ret sequence under operation of a third cognate promoter.

Aspects of the present disclosure are directed to a nucleic acid storage system including an engineered, non-naturally occurring cell including one or more nucleic acid sequences encoding a Cas1 protein and/or a Cas2 protein of a CRISPR adaptation system, a CRISPR array nucleic acid sequence including a leader sequence and at least one repeat sequence, wherein the CRISPR array nucleic acid sequence is within genomic DNA of the cell or on a plasmid, a retron system which is used to produce different protospacer DNA sequences to be introduced into the CRISPR array, wherein the retron system includes (1) a first nucleic acid sequence comprising a first msd sequence 5′ to an msr sequence wherein the first msd sequence is proximal to and under operation of a first cognate promoter and further including a first complementary sequence between the first cognate promoter and the first msd sequence, (2) a second nucleic acid sequence comprising a second msd sequence 5′ to an msr sequence wherein the second msd sequence is proximal to and under operation of a second cognate promoter and further including a second complementary sequence between the second cognate promoter and the second msd sequence, wherein the first msd sequence is different from the second msd sequence and wherein the first complementary sequence and the second complementary sequence are complementary to each other, and (3) a third nucleic acid comprising a ret sequence under operation of a third cognate promoter. According to one aspect, at least three protospacer DNA sequences are generated by the retron system and are processed and spacer sequences are inserted into the CRISPR array nucleic acid sequence.

Aspects of the present disclosure are directed to system for in vivo molecular recording including an engineered, non-naturally occurring cell including one or more nucleic acid sequences encoding a Cas1 protein and/or a Cas2 protein of a CRISPR adaptation system, a CRISPR array nucleic acid sequence including a leader sequence and at least one repeat sequence, wherein the CRISPR array nucleic acid sequence is within genomic DNA of the cell or on a plasmid, a retron system which is used to produce different protospacer DNA sequences to be introduced into the CRISPR array, wherein the retron system includes (1) a first nucleic acid sequence comprising a first msd sequence 5′ to an msr sequence wherein the first msd sequence is proximal to and under operation of a first cognate promoter and further including a first complementary sequence between the first cognate promoter and the first msd sequence, (2) a second nucleic acid sequence comprising a second msd sequence 5′ to an msr sequence wherein the second msd sequence is proximal to and under operation of a second cognate promoter and further including a second complementary sequence between the second cognate promoter and the second msd sequence, wherein the first msd sequence is different from the second msd sequence and wherein the first complementary sequence and the second complementary sequence are complementary to each other, and (3) a third nucleic acid comprising a ret sequence under operation of a third cognate promoter. According to one aspect, at least three protospacer DNA sequences are generated by the retron system and are processed and spacer sequences are inserted into the CRISPR array nucleic acid sequence.

Aspects of the present disclosure are directed to a kit for in vivo molecular recording including in a first container, an engineered, non-naturally occurring cell including one or more nucleic acid sequences encoding a Cas1 protein and/or a Cas2 protein of a CRISPR adaptation system, a CRISPR array nucleic acid sequence including a leader sequence and at least one repeat sequence wherein the CRISPR array nucleic acid sequence is within genomic DNA of the cell or on a plasmid, in a second container, a retron system which is used to produce different protospacer DNA sequences to be introduced into the CRISPR array, wherein the retron system includes (1) a first nucleic acid sequence comprising a first msd sequence 5′ to an msr sequence wherein the first msd sequence is proximal to and under operation of a first cognate promoter and further including a first complementary sequence between the first cognate promoter and the first msd sequence, (2) a second nucleic acid sequence comprising a second msd sequence 5′ to an msr sequence wherein the second msd sequence is proximal to and under operation of a second cognate promoter and further including a second complementary sequence between the second cognate promoter and the second msd sequence, wherein the first msd sequence is different from the second msd sequence and wherein the first complementary sequence and the second complementary sequence are complementary to each other, and (3) a third nucleic acid comprising a ret sequence under operation of a third cognate promoter, and optional instructions for use.

What is claimed is:

1. A method of altering a cell comprising
providing the cell with one or more nucleic acid sequences encoding a Cas1 protein and/or a Cas2 protein of a CRISPR adaptation system,
providing the cell with a CRISPR array nucleic acid sequence including a leader sequence and at least one repeat sequence, wherein the CRISPR array nucleic acid sequence is within genomic DNA of the cell or on a plasmid,
providing the cell with one or more retron systems which are used to produce protospacer DNA sequences to be introduced into the CRISPR array,
wherein the cell expresses the Cas1 protein and/or the Cas2 protein,
wherein the retron system produces the protospacer DNA sequence, and
wherein the protospacer DNA sequence is processed and a spacer sequence is inserted into the CRISPR array nucleic acid sequence.

2. The method of claim 1 wherein the protospacer is a defined synthetic DNA.

3. The method of claim 2 wherein the protospacer sequence includes a modified “AAG” protospacer adjacent motif (PAM).

4. The method of claim 1 wherein the nucleic acid sequence encoding the Cas1 protein and/or a Cas2 protein is provided to the cell within a vector.

5. The method of claim 1 wherein the retron system is provided to the cell within a vector.

6. The method of claim 1 wherein the cell is a prokaryotic or a eukaryotic cell.

7. The method of claim 1 wherein the nucleic acid sequence encoding the Cas1 protein and/or a Cas2 protein comprises inducible promoters for induction of expression of the Cas1 and/or Cas2 protein.

8. An engineered, non-naturally occurring cell comprising
one or more nucleic acid sequences encoding a Cas1 protein and/or a Cas2 protein of a CRISPR adaptation system,
a CRISPR array nucleic acid sequence including a leader sequence and at least one repeat sequence, and
one or more retron systems which are used to produce protospacer DNA sequences to be introduced into the CRISPR array,
wherein the CRISPR array nucleic acid sequence is within genomic DNA of the cell or on a plasmid, and
wherein the cell expresses the Cas1 protein and/or the Cas 2 protein.

9. The engineered, non-naturally occurring cell of claim 8 including at least one spacer sequence inserted into the CRISPR array nucleic acid sequence, which spacer sequence was derived from a corresponding protospacer sequence generated by the one or more retron systems.

10. A method of inserting a target DNA sequence within genomic DNA of a cell comprising
generating the target DNA sequence within the cell using one or more exogenous retron systems, wherein the cell includes a nucleic acid sequence encoding a Cas1 protein and/or a Cas2 protein of a CRISPR adaptation system and a CRISPR array nucleic acid sequence including a leader sequence and at least one repeat sequence,
wherein the cell expresses the Cas1 protein and/or the Cas2 protein and wherein the CRISPR array nucleic acid sequence is within genomic DNA of the cell or on a plasmid, and
wherein the target DNA sequence is generated under conditions within the cell wherein the Cas1 protein and/or the Cas2 protein processes the target DNA sequence and the target DNA sequence is inserted into the CRISPR array nucleic acid sequence adjacent a corresponding repeat sequence.

11. The method of claim 10 wherein the target DNA sequence is a protospacer.

12. The method of claim 10 wherein the target DNA sequence is a defined synthetic protospacer DNA sequence.

13. The method of claim 10 wherein the target DNA sequence includes a modified “AAG” protospacer adjacent motif (PAM).

14. The method of claim 10 wherein the step of generating is repeated such that a plurality of target DNA sequences are inserted into the CRISPR array nucleic acid sequence at corresponding repeat sequences.

15. The method of claim 10 wherein the nucleic acid sequence encoding the Cas1 protein and/or a Cas2 protein is provided to the cell within a vector.

16. The method of claim 10 wherein the cell is a prokaryotic or a eukaryotic cell.

17. A nucleic acid storage system comprising
an engineered, non-naturally occurring cell including
one or more nucleic acid sequences encoding a Cas1 protein and/or a Cas2 protein of a CRISPR adaptation system,
a CRISPR array nucleic acid sequence including a leader sequence and at least one repeat sequence, and
one or more retron systems which are used to produce protospacer DNA sequences to be processed and introduced into the CRISPR array,
wherein the CRISPR array nucleic acid sequence is within genomic DNA of the cell or on a plasmid, and
wherein the cell expresses the Cas1 protein and/or the Cas 2 protein.

18. The nucleic acid storage system of claim 17 wherein at least one protospacer DNA sequence is generated by the one or more retron systems and is processed and a spacer sequence is inserted into the CRISPR array nucleic acid sequence.

19. A system for in vivo molecular recording comprising
an engineered, non-naturally occurring cell including
one or more nucleic acid sequences encoding a Cas1 protein and/or a Cas2 protein of a CRISPR adaptation system,
a CRISPR array nucleic acid sequence including a leader sequence and at least one repeat sequence, and
one or more retron systems which are used to produce protospacer DNA sequences to be processed and introduced into the CRISPR array,
wherein the CRISPR array nucleic acid sequence is within genomic DNA of the cell or on a plasmid, and
wherein the cell expresses the Cas1 protein and/or the Cas 2 protein.

20. A kit for in vivo molecular recording comprising
in a first container, an engineered, non-naturally occurring cell including
one or more nucleic acid sequences encoding a Cas1 protein and/or a Cas2 protein of a CRISPR adaptation system,
a CRISPR array nucleic acid sequence including a leader sequence and at least one repeat sequence wherein the CRISPR array nucleic acid sequence is within genomic DNA of the cell or on a plasmid,
in a second container, one or more retron systems to be supplied to the cell which are used to produce protospacer DNA sequences to be processed and introduced into the CRISPR array, and
optional instructions for use.

21. The method of claim 1 further comprising providing the cell with a plurality of retron systems which are used to produce different protospacer DNA sequences to be introduced into the CRISPR array,
wherein the plurality of retron systems produce the different protospacer DNA sequences, and
wherein the different protospacer DNA sequences are processed and spacer sequences are inserted into the CRISPR array nucleic acid sequence.

22. The method of claim 1 wherein the retron system includes a first nucleic acid sequence comprising an msr sequence and an msd sequence under operation of a first cognate promoter and a second nucleic acid sequence comprising a ret sequence under operation of a second cognate promoter.

23. The method of claim 1 wherein the retron system includes a first nucleic acid sequence comprising an msr sequence under operation of a first cognate promoter, a second nucleic acid sequence comprising an msd sequence under operation of a second cognate promoter and a third nucleic acid sequence comprising a ret sequence under operation of a third cognate promoter.

24. The method of claim 1 wherein the retron system includes a first nucleic acid sequence comprising an msr sequence under operation of a first cognate promoter, a second nucleic acid sequence comprising an msd sequence under operation of a second cognate promoter and a third nucleic acid sequence comprising a ret sequence under operation of a third cognate promoter,
wherein the second nucleic acid sequence includes an additional DNA sequence between the second cognate promoter and the msd sequence which is transcribed with the msd sequence.

25. The method of claim 1 further comprising providing the cell with a plurality of retron systems which are used to produce different protospacer DNA sequences to be introduced into the CRISPR array,
wherein the plurality of retron systems produce the different protospacer DNA sequences, and
wherein the different protospacer DNA sequences are processed and spacer sequences are inserted into the CRISPR array nucleic acid sequence,
wherein each retron system of the plurality includes a first nucleic acid sequence comprising an msr sequence and an msd sequence under operation of a first cognate promoter and a second nucleic acid sequence comprising a ret sequence under operation of a second cognate promoter.

26. The method of claim 25 wherein the first cognate promoter of each retron system is separately inducible.

27. The method of claim 25 wherein the first cognate promoter of each retron system is separately inducible simultaneously or nonsimultaneously.

28. The method of claim 1 further comprising providing the cell with a plurality of retron systems which are used to produce different protospacer DNA sequences to be introduced into the CRISPR array,
wherein the plurality of retron systems produce the different protospacer DNA sequences, and
wherein the different protospacer DNA sequences are processed and spacer sequences are inserted into the CRISPR array nucleic acid sequence,
wherein each retron system of the plurality includes a first nucleic acid sequence comprising an msr sequenced under operation of a first cognate promoter, a second nucleic acid sequence comprising an msd sequence under operation of a second cognate promoter and a third nucleic acid sequence comprising a ret sequence under operation of a third cognate promoter.

29. The method of claim 28 wherein the second cognate promoter of each retron system is separately inducible.

30. The method of claim 28 wherein the second cognate promoter of each retron system is separately inducible simultaneously or nonsimultaneously.

31. The method of claim 28 wherein the second nucleic acid sequence includes an additional DNA sequence between the second cognate promoter and the msd sequence which is transcribed with the msd sequence.

32. The engineered, non-naturally occurring cell of claim 8 further comprising
a plurality of retron systems which are used to produce different protospacer DNA sequences to be introduced into the CRISPR array.

33. The method of claim 10 further comprising inserting a plurality of different target DNA sequences within genomic DNA of a cell wherein the plurality of different target DNA sequences are generated within the cell using a plurality of exogenous retron systems, and
wherein the Cas1 protein and/or the Cas2 protein processes the plurality of different target DNA sequences and the plurality of different target DNA sequences are inserted into the CRISPR array nucleic acid sequence adjacent a corresponding repeat sequence.

34. The nucleic acid storage system of claim 17 further comprising
a plurality of retron systems which are used to produce different protospacer DNA sequences to be processed and introduced into the CRISPR array.

35. The system for in vivo molecular recording of claim 19 further comprising
a plurality of retron systems which are used to produce different protospacer DNA sequences to be processed and introduced into the CRISPR array.

36. The kit of claim 20 further comprising
in the second container, a plurality of retron systems to be supplied to the cell which are used to produce different protospacer DNA sequences to be processed and introduced into the CRISPR array.

37. A method of altering a cell comprising
providing the cell with one or more nucleic acid sequences encoding a Cas1 protein and/or a Cas2 protein of a CRISPR adaptation system,
providing the cell with a CRISPR array nucleic acid sequence including a leader sequence and at least one repeat sequence, wherein the CRISPR array nucleic acid sequence is within genomic DNA of the cell or on a plasmid,
providing the cell with a retron system which is used to produce different protospacer DNA sequences to be introduced into the CRISPR array,
wherein the retron system includes (1) a first nucleic acid sequence comprising a first msd sequence 5′ to an msr sequence wherein the first msd sequence is proximal to and under operation of a first cognate promoter and further including a first complementary sequence between the first cognate promoter and the first msd sequence, (2) a second nucleic acid sequence comprising a second msd sequence 5′ to an msr sequence wherein the second msd sequence is proximal to and under operation of a second cognate promoter and further including a second complementary sequence between the second cognate promoter and the second msd sequence, wherein the first msd sequence is different from the second msd sequence and wherein the first complementary sequence and the second complementary sequence are complementary to each other, and (3) a third nucleic acid comprising a ret sequence under operation of a third cognate promoter
wherein the cell expresses the Cas1 protein and/or the Cas2 protein,
wherein the retron system produces a first protospacer DNA sequence corresponding to the first msd sequence, a second protospacer DNA sequence corresponding to the second msd sequence, and a third protospacer sequence corresponding to the first complementary sequence and the second complementary sequence hybridized to each other,
wherein the first, second and third protospacer DNA sequences are processed and spacer sequences are inserted into the CRISPR array nucleic acid sequence.

38. The method of claim 37 wherein the first cognate promoter and the second cognate promoter of the retron system are separately inducible.

39. The method of claim 37 wherein the first cognate promoter and the second cognate promoter of the retron system are separately inducible simultaneously or nonsimultaneously.

40. The method of claim 37 wherein the first, second and third protospacer DNA sequences are defined synthetic DNA.

41. The method of claim 37 wherein the first, second and third protospacer DNA sequences include a modified “AAG” protospacer adjacent motif (PAM).

42. The method of claim 37 wherein the one or more nucleic acid sequences encoding the Cas1 protein and/or a Cas2 protein is provided to the cell within a vector.

43. The method of claim 37 wherein the retron system is provided to the cell within a vector.

44. The method of claim 37 wherein the cell is a prokaryotic or a eukaryotic cell.

45. An engineered, non-naturally occurring cell comprising
one or more nucleic acid sequences encoding a Cas1 protein and/or a Cas2 protein of a CRISPR adaptation system,
a CRISPR array nucleic acid sequence including a leader sequence and at least one repeat sequence, wherein the CRISPR array nucleic acid sequence is within genomic DNA of the cell or on a plasmid,
a retron system which is used to produce different protospacer DNA sequences to be introduced into the CRISPR array,
wherein the retron system includes (1) a first nucleic acid sequence comprising a first msd sequence 5′ to an msr sequence wherein the first msd sequence is proximal to and under operation of a first cognate promoter and further including a first complementary sequence between the first cognate promoter and the first msd sequence, (2) a second nucleic acid sequence comprising a second msd sequence 5′ to an msr sequence wherein the second msd sequence is proximal to and under operation of a second cognate promoter and further including a second complementary sequence between the second cognate promoter and the second msd sequence, wherein the first msd sequence is different from the second msd sequence and wherein the first complementary sequence and the second complementary sequence are complementary to each other, and (3) a third nucleic acid comprising a ret sequence under operation of a third cognate promoter.

46. A nucleic acid storage system comprising
an engineered, non-naturally occurring cell comprising
one or more nucleic acid sequences encoding a Cas1 protein and/or a Cas2 protein of a CRISPR adaptation system,
a CRISPR array nucleic acid sequence including a leader sequence and at least one repeat sequence, wherein the CRISPR array nucleic acid sequence is within genomic DNA of the cell or on a plasmid,
a retron system which is used to produce different protospacer DNA sequences to be introduced into the CRISPR array,
wherein the retron system includes (1) a first nucleic acid sequence comprising a first msd sequence 5′ to an msr sequence wherein the first msd sequence is proximal to and under operation of a first cognate promoter and further including a first complementary sequence between the first cognate promoter and the first msd sequence, (2) a second nucleic acid sequence comprising a second msd sequence 5′ to an msr sequence wherein the second msd sequence is proximal to and under operation of a second cognate promoter and further including a second complementary sequence between the second cognate promoter and the second msd sequence, wherein the first msd sequence is different from the second msd sequence and wherein the first complementary sequence and the second complementary sequence are complementary to each other, and (3) a third nucleic acid comprising a ret sequence under operation of a third cognate promoter.

47. The nucleic acid storage system of claim 46 wherein at least three protospacer DNA sequences are generated by the retron system and are processed and spacer sequences are inserted into the CRISPR array nucleic acid sequence.

48. A system for in vivo molecular recording comprising
an engineered, non-naturally occurring cell comprising
one or more nucleic acid sequences encoding a Cas1 protein and/or a Cas2 protein of a CRISPR adaptation system,
a CRISPR array nucleic acid sequence including a leader sequence and at least one repeat sequence, wherein the CRISPR array nucleic acid sequence is within genomic DNA of the cell or on a plasmid,
a retron system which is used to produce different protospacer DNA sequences to be introduced into the CRISPR array,
wherein the retron system includes (1) a first nucleic acid sequence comprising a first msd sequence 5′ to an msr sequence wherein the first msd sequence is proximal to and under operation of a first cognate promoter and further including a first complementary sequence between the first cognate promoter and the first msd sequence, (2) a second nucleic acid sequence comprising a second msd sequence 5′ to an msr sequence wherein the second msd sequence is proximal to and under operation of a second cognate promoter and further including a second complementary sequence between the second cognate promoter and the second msd sequence, wherein the first msd sequence is different from the second msd sequence and wherein the first complementary sequence and the second complementary sequence are complementary to each other, and (3) a third nucleic acid comprising a ret sequence under operation of a third cognate promoter.

49. The nucleic acid storage system of claim 48 wherein at least three protospacer DNA sequences are generated by the retron system and are processed and spacer sequences are inserted into the CRISPR array nucleic acid sequence.

50. A kit for in vivo molecular recording comprising
in a first container, an engineered, non-naturally occurring cell including
one or more nucleic acid sequences encoding a Cas1 protein and/or a Cas2 protein of a CRISPR adaptation system,
a CRISPR array nucleic acid sequence including a leader sequence and at least one repeat sequence wherein the CRISPR array nucleic acid sequence is within genomic DNA of the cell or on a plasmid,
in a second container, a retron system which is used to produce different protospacer DNA sequences to be introduced into the CRISPR array,
wherein the retron system includes (1) a first nucleic acid sequence comprising a first msd sequence 5′ to an msr sequence wherein the first msd sequence is proximal to and under operation of a first cognate promoter and further including a first complementary sequence between the first cognate promoter and the first msd sequence, (2) a second nucleic acid sequence comprising a second msd sequence 5′ to an msr sequence wherein the second msd sequence is proximal to and under operation of a second cognate promoter and further including a second complementary sequence between the second cognate promoter and the second msd sequence, wherein the first msd sequence is different from the second msd sequence and wherein the first complementary sequence and the second complementary sequence are complementary to each other, and (3) a third nucleic acid comprising a ret sequence under operation of a third cognate promoter, and
optional instructions for use.
